(19) United States
(12) Patent Application Publication
(10) Pub. No.: US 2024/0101698 A1
(43) Pub. Date: Mar. 28, 2024

(54) FORMULATIONS OF ANTI-CD73 ANTIBODIES

(71) Applicant: I-MAB BIOPHARMA CO.,LTD, Gaithersburg, MD (US)

(72) Inventors: Jing ZHU, Shanghai (CN); Zheru ZHANG, Shanghai (CN); Xiangping ZHU, Shanghai (CN); Yali YU, Shanghai (CN)

(21) Appl. No.: 18/270,472

(22) PCT Filed: May 8, 2021
(86) PCT No.: PCT/CN2021/092485

§ 371 Date: Jun. 29, 2023

(30) Foreign Application Priority Data
Dec. 30, 2020 (WO) PCT/CN2020/141601

(51) Int. Cl. C07K 16/28; A61K 9/19; A61K 47/22; A61K 47/26

(52) U.S. Cl. CPC C07K 16/2896; A61K 9/19; A61K 47/22; A61K 47/26; A61K 2039/505

(57) ABSTRACT

Provided are stable formulations of anti-CD73 antibodies, which include 5-50 mM histidine, 2%-20% (w/v) trehalose, and 0.015%-0.05% (w/v) polysorbate 80 (PS80), at pH 5.6-6.4. Also provided are lyophilized compositions that can be obtained by drying the aqueous formulations and methods for treating diseases.

### BACKGROUND

Anti-CD73 antibodies are being developed for treating various proliferative and inflammatory diseases. CD73 (cluster of differentiation 73) is an enzyme serves to convert AMP to adenosine. CD73 catalyzes the formation of extracellular adenosine which contributes to the immunosuppressive tumor environment. CD73 is over-expressed in stromal cells and multiple types of tumor cells, as well as in Tregs, M2 Mφs and myeloid derived suppressor cells (MDSCs).

CD73 inhibition prevented adenosine-mediated lymphocyte suppression, increased the activity of CD8+ effector cells, and reduced both MDSCs and Tregs. There are a few anti-CD73 antibodies being developed as potential anticancer agents, but none have been approved for clinical use.

Delivery by injection is generally the delivery route of choice for cancer treatments with antibodies or antigen binding fragments. However, biological, chemical, and physical barriers such as poor long-term storage, osmolality, solubility, and stability make delivery of biologically active agents by injection to mammals problematic. Therefore, there exists a need for improved injectable preparations of antibodies, which are stable and soluble.

### SUMMARY

The present disclosure provides a composition comprising an anti-CD73 antibody, 5-50 mM histidine, 2%-20% (w/v) trehalose, and 0.015%-0.05% (w/v) polysorbate 80 (PS80), at pH 5.6-6.4, wherein the antibody comprises a heavy chain variable region (VH) comprising a CDR1, a CDR2 and a CDR3, and a light chain variable region (VL) comprising a CDR1, a CDR2 and a CDR3, and wherein the VH CDR1, VH CDR2, VH CDR3, VL CDR1, VL CDR2, and VL CDR3 comprise the amino acid sequences of SEQ ID NO: 1-6, respectively.

In some embodiments, the composition comprises 10-30 histidine, 5%-15% trehalose, and 0.015%-0.035% (w/v) PS80, at pH 5.8-6.2. In some embodiments, the composition comprises 15-25 mM histidine, 6%-10% (w/v) trehalose, 0.015%-0.025% (w/v) PS80, at pH 5.9-6.1. In some embodiments, the composition comprises 5-150 mg/mL of the antibody.

In some embodiments, the VH comprises the amino acid sequence of SEQ ID NO: 7, and the VL comprises the amino acid sequence of SEQ ID NO: 8. In some embodiments, the antibody comprises a heavy chain comprising the amino acid sequence of SEQ ID NO: 9, and a light chain comprising the amino acid sequence of SEQ ID NO: 10.

Also provided, in one embodiment, is a lyophilized composition obtainable by freeze-drying the composition of the disclosure. Also provided is a solution (e.g., water) obtainable by dissolving the lyophilized composition.

Also provided are uses and methods of using the composition for treating cancer, such as bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer.

### DETAILED DESCRIPTION

### I. Definitions

All numerical designations, e.g., pH, temperature, time, concentration, and molecular weight, including ranges, are approximations which are varied (+) or (−) by increments of 0.1 or 10%. It is to be understood, although not always explicitly stated that all numerical designations are preceded by the term “about”. It also is to be understood, although not always explicitly stated, that the reagents described herein are merely exemplary and that equivalents of such are known in the art.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

### II. Antibody Formulations

Development of a suitable formation for a therapeutic antibody typically has conflicting requirements from, e.g., protein solubility, stability, and osmolality. It is therefore challenging and unpredictable whether such requirements could be balanced to generate an acceptable formation. For instance, as demonstrated in Example 2, when different buffers were tested with the anti-CD73 antibody of the present disclosure, the phosphate buffer appeared to allow more fragmentation of the antibody to occur (see, e.g., Table 4) than other buffers. It also led to more pronounced reduction of purity (Table 5). These data also suggest that high (pH 7.0) and low (pH 4.5) pH conditions can also be detrimental to the purity of the protein (see also Table 6).

Among all these tested buffers and conditions, formulation F5 with 20 nM histidine at pH 6.0 appeared to be able to support the stability of the protein. Surprisingly, this buffer condition also allowed for high solubility of the protein, for at least 80 mg/mL and 150 mg/mL (Table 7).

A number of candidate excipients were tested in Example 3, including sucrose, trehalose, sorbitol, mannitol, and arginine. Surprisingly, visible particles were detected in the formulation that contained mannitol, one of the most commonly used excipients for protein formulation, following freeze-thaw testing, even in the presence of PS80 (polysorbate 80), an excipient identified as important for stabilizing the protein during freeze-thaw cycles (Tables 11-13). Also, the formulation with sucrose suffered the most fragmentation (Table 17). Use of another excipient, arginine, also led to relatively large (1%) decrease of protein purity (Table 18). Yet another excipient, sorbitol, failed to sufficiently support protein stability during 10 cycles of freeze-thaw (Table 23).

Interestingly, one of the candidate excipients, trehalose, was able to keep the protein sufficiently stable through all these testing conditions. Also, the formulation with trehalose maintained the antibody's antigen-binding potency (Table 24). Then, in Example 5, the thermodynamic stability of the formulations was tested and it was found that at least 0.02% PS80 was required to keep the protein stable during the testing procedure. Therefore, through trial and error, the instant inventors were able to identify a suitable formulation for the anti-CD73 antibody that includes histidine, trehalose, and PS80 at about pH 6.0.

In accordance with one embodiment of the present disclosure, provided is a composition that includes anti-CD73 antibody or antigen-binding fragment of the present disclosure, histidine, trehalose, and polysorbate 80. In some embodiments, the composition is an aqueous solution and the pH of the solution is 5.5 to 6.5.

In some embodiments, histidine (e.g., histidine HCl) is present at a concentration of about 5-50 mM. In some embodiments, histidine's concentration is at least about 5 mM, 6 mM, 7 mM, 8 mM, 9 mM, 10 mM, 11 mM, 12 MM, 13 mM, 14 mM, 15 mM, 16 mM, 17 mM, 18 mM, 19 mM, 20 mM 25 mM, 30 mM, 35 mM, 40 mM, or 45 mM. In some embodiments, histidine's concentration is not higher than about 100 mM, 90 mM, 80 mM, 70 mM, 60 mM, 50 mM, 45 mM, 40 mM, 35 mM, 30 mM, 29 mM, 28 mM, 27 mM, 26 mM, 25 mM, 24 mM, 23 mM, 22 mM, 21 mM, 20 mM, 19 mM, 18 mM, 17 mM, 16 mM, or 15 mM. In some embodiments, histidine's concentration is about 5-40 mM, 5-35 mM, 5-30 mM, 5-25 mM, 5-20 mM, 10-50 mM, 10-45 mM, 10-40 mM, 10-35 mM, 10-30 mM, 10-25 mM, 10-20 mM, 15-50 mM, 15-45 mM, 15-40 mM, 15-35 mM, 15-30 mM, 15-25 mM, 15-20 mM, 20-50 mM, 20-45 mM, 20-40 mM, 20-35 mM, 20-30 mM, or 20-25 mM.

In some embodiments, trehalose (e.g., trehalose dihydrate) is present at a concentration of about 2%-20% (w/v). In some embodiments, trehalose's concentration is at least about 2%, 3%, 4%, 5%, 6%, 7% 8%, 9%, 10%, 11%, 12%, 15%, 17% or 18% (w/v). In some embodiments, trehalose's concentration is not higher than about 20%, 19%, 18%, 17%, 16%, 15%, 14%, 13%, 12%, 11%, 10%, 9%, 8%, 7%, 6%, 5%, 4%, or 3% (w/v). In some embodiments, trehalose's concentration is about 2%-20%, 2%-19%, 2%-18%, 2%-17%, 2%-16%, 2%-15%, 2%-14%, 2%-13%, 2%-12%, 2%-11%, 2%-10%, 2%-9%, 2%-8%, 3%-20%, 3%-19%, 3%-18%, 3%-17%, 3%-16%, 3%-15%, 3%-14%, 3%-13%, 3%-13%, 3%-11%, 3%-10%, 3%-9%, 3%-8%, 4%-20%, 4%-19%, 4%-18%, 4%-17%, 4%-16%, 4%-15%, 4%-14%, 4%-13%.4%-14%, 4%-11%, 4%-10%, 4%-9%, 4%-8%, 5%-20%, 5%-19%, 5%-18%, 5%-17%, 5%-16%, 5%-15%, 5%-14%, 5%-13%, 5%-15%, 5%-1. 1%, 5%-10%, 5%-8%, 6%-20%, 6%-19%, 6%-18%, 6%-17%, 6%-16%, 6%-16%, 6%-14%, 6%-13%, 6%-16%, 6%-11%, 6%-10%, 6%-9%, 6%-8%, 7%-20%, 7%-19%, 7%-18%, 7%-17%, 7%-17%, 7%-17%, 7%-14%, 7%-13%, 7%-17%, 7%-11%, 7%-10%, 7%-9%, 7%-8%, 8%-20%, 8%-19%, 8%-18%, 8%-18%, 8%-18%, 8%-18%, 8%-14%, 8%-13%, 8%-18%, 8%-11%, 8%-10%, 8%-9%, 9%-20%, 9%-19%, 9%-18%, 9%-19%, 9%-19%, 9%-19%, 9%-14%, 9%-13%, 9%-19%, 9%-11%, 9%-10%, 10%, 4-20%, 10%-19%, 10%-18%, 10%-110%, 10%-110%, 10%-110%, 10%-14%, 10%-13%, 10%-110%, or 10%-11% (w/v).

In some embodiments, polysorbate 80 (PS80) is present at a concentration of about 0.015%-0.05% (w/v). In some embodiments, PS80 is present at a concentration of at least about 0.015%, 0.016%, 0.017%, 0.018%, 0.019%, 0.02%, 0.021%, 0.022%, 0.023%, 0.024%, 0.025%, 0.026%, 0.027%, 0.028%, 0.029%, or 0.03% (w/v). In some embodiments, PS80 is present at a concentration of not higher than 0.05%, 0.049%, 0.048%, 0.047%, 0.046%, 0.045%, 0.044%, 0.043%, 0.042%, 0.041%, 0.04%, 0.039%, 0.038%, 0.037%, 0.036%, 0.035%, 0.034%, 0.033%, 0.032%, 0.031%, 0.03%, 0.029%, 0.028%, 0.027%, 0.026%, 0.025%, 0.024%, 0.023%, 0.022%, 0.021%, or 0.02% (w/v).

In some embodiments, the composition has a pH of 5.6-6.4. In some embodiments, the pH is not lower than 5.6, 5.7, 5.8, 5.85, 5.9, 5.95, 6, 6.05, 6.1, 6.15, or 6.2. In some embodiments, the pH is not higher than 6.4, 6,3, 6.2, 6.15, 6,1, 6.05, 6, 5.95, 5,9, 5.85, or 5.8. In some embodiments, the pH is about 5,7-6.3, 5.8-6.2, 5.85-6,15, 5.9-6.1, 5,95-6.05, or at about 5.9, 5.95, 6, 6.05, or 6.1.

In some embodiments, the composition further includes one or more bulking agents. As used herein, the term “bulking agent” refers to an ingredient that provides bulk to a lyophilized formulation. Examples of bulking agents include, without limitation, mannitol, trehalose, lactose, sucrose, polyvinyl pyrrolidone, sucrose, glucose, glycine, cyclodextrins, dextran, solid PEGS and derivatives and mixtures thereof. In one embodiment, a formulation of the present disclosure optionally includes a bulking agent.

In some embodiments, the composition further includes one or more tonicity agents. The term “tonicity agent” as used herein denotes pharmaceutically acceptable agents used to modulate the tonicity of the formulation. Isotonicity generally relates to the osmotic pressure relative to a solution, usually relative to that of human blood serum. A formulation can be hypotonic, isotonic or hypertonic. In one aspect, the formulation is isotonic. An isotonic formulation is liquid or liquid reconstituted from a solid form, e.g. from a lyophilized form and denotes a solution having the same tonicity as some other solution with which it is compared, such as physiologic salt solution and the blood serum. Suitable isotonicity agents include but are not limited to sodium chloride, potassium chloride, glycerin and any component from the group of amino acids, sugars, as defined herein as well as combinations thereof.

In some embodiments, the composition further includes one or more surfactants. As used herein, the term “surfactant” refers to a pharmaceutically acceptable organic substance having amphipathic structures; namely, it is composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials. In some embodiments of the pharmaceutical formulations described herein, the amount of surfactant is described as a percentage expressed in weight/volume percent (w/v %). Suitable pharmaceutically acceptable surfactants include but are not limited to the group of polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), or sodium dodecyl sulphate (SDS). Polyoxyethylenesorbitan-fatty acid esters include polysorbate 20, (sold under the trademark Tween 20™) and polysorbate 80 (sold under the trademark Tween 80™). Polyethylene-polypropylene copolymers include those sold under the names Pluronic® F68 or Poloxamer 188™. Polyoxyethylene alkyl ethers include those sold under the trademark Brij™. Alkylphenolpolyoxyethylene ethers include those sold under the tradename Triton-X.

In some embodiments, the composition further includes one or more lyoprotectants. A “lyoprotectant” refers to a pharmaceutically acceptable substance that stabilizes a protein during lyophilization (the process of rapid freezing and drying in a high vacuum). Examples of lyoprotectants include, without limitation, sucrose, trehalose or mannitol.

In some embodiments, the composition further includes one or more antioxidants. An “antioxidant” refers to a molecule capable of slowing or preventing the oxidation of other molecules. Oxidation is a chemical reaction that transfers electrons from a substance to an oxidizing agent. Oxidation reactions can produce free radicals, which start chain reactions that destabilize the protein therapeutics and ultimately affect the product activity. Antioxidants terminate these chain reactions by removing free radical intermediates, and inhibit other oxidation reactions by being oxidized themselves. As a result, antioxidants are often reducing agents, chelating agent and oxygen scavengers such as citrate, EDTA, DPTA, thiols, ascorbic acid or polyphenols. Non-limiting examples of antioxidants include ascorbic acid (AA, E300), thiosulfate, methionine, tocopherols (E306), propyl gallate (PG, E310), tertiary butylhydroquinone (TBHQ), butylated hydroxyanisole (BHA, E320) and butylated hydroxytoluene (BHT, E321).

In some embodiments, the composition further includes one or more preservatives. A “preservative” is a natural or synthetic chemical that is added to products such as foods, pharmaceuticals, paints, biological samples, wood, etc. to prevent decomposition by microbial growth or by undesirable chemical changes. Preservative additives can be used alone or in conjunction with other methods of preservation: Preservatives may be antimicrobial preservatives, which inhibit the growth of bacteria and fungi, or antioxidants such as oxygen absorbers, which inhibit the oxidation of constituents. Common antimicrobial preservatives include, benzalkonium chloride, benzoic acid, cholorohexidine, glycerin, phenol, potassium sorbate, thimerosal, sulfites (sulfur dioxide, sodium bisulfite, potassium hydrogen sulfite, etc.) and disodium EDTA. Other preservatives include those commonly used in patenteral proteins such as benzyl alcohol, phenol, m-cresol, chlorobutanol or methylparaben.

The anti-CD73 antibodies, as disclosed herein, have a heavy chain variable (VH) region and a light chain variable (VL) region. The VII includes three complementarity determining regions (CDR), CDR1, CDR2 and CDR3 and the VL also includes three CDRs, CDR1, CDR2 and CDR3. The VH CDR1, CDR2 and CDR3 and VL CDR1, CDR2 and CDR3 include the amino acid sequences SEQ ID NO: 1-6, respectively. Examples \If and VL sequences include SEQ ID NO: 7 and 8, respectively. The antibody can also include heavy chain and light chain constant regions. Example heavy chain and light chain sequences include SEQ ID NO: 9 and 10, respectively.

In some embodiments, the composition includes 5-150 mg/mL of the anti-CD73 antibody or antigen-binding fragment of the disclosure, 5-50 histidine, 2%-20% (w/v) trehalose, 0.015%-0.05% (w/v) PS80, at pH 5.6-6.4.

In some embodiments, the composition includes 10-12.0 mg/mL of the anti-CD73 antibody or antigen-binding fragment of the disclosure, 10-30 mM histidine, 5%-15% (w/v) trehalose, 0.015%-0.035% (w/v) PS80, at pH 5.8-6.2.

In some embodiments, the composition includes 10-120 mg/mL of the anti-CD73 antibody or antigen-binding fragment of the disclosure, 15-25 mM histidine, 6%-10% (w/v) trehalose, 0.015%-0.025% (w/v) PS80, at pH 5.9-6.1.

Also provided, in some embodiments, is a lyophilized composition that can be prepared by freeze-drying the aqueous solution as disclosed herein. In some embodiments, also provided is a solution that can be prepared by dissolving the lyophilized composition in a solvent such as water.

### III. Methods of Using the Formulations

As described herein, the compositions of the present disclosure may be used in certain treatment and diagnostic methods. The present disclosure is further directed to antibody-based therapies which involve administering the composition of the disclosure to a patient such as an animal, a mammal, and a human for treating one or more of the disorders or conditions described. herein.

The compositions of the disclosure can also be used to treat or inhibit cancer. As provided above, CD73 can be overexpressed in tumor cells. Tumor-derived CD73 can function as an ecto-enzyme to produce extracellular adenosine, which promotes tumor growth by limiting antitumor T-cell immunity via adenosine receptor signaling. Results with small molecule inhibitors, or monoclonal antibodies targeting CD73 in murine tumor models, indicate that targeted CD73 therapy is an important alternative and realistic approach to effective control of tumor growth. In particular, it helps T-cell-based therapy by enhancing the adaptive immune response machinery, which may increase the function of tumor-infiltrating T lymphocytes, and lead to improved survival in cancer patients.

Accordingly, in some embodiments, provided are methods for treating a cancer in a patient in need thereof. The method, in one embodiment, entails administering to the patient an effective amount of a composition of the present disclosure. In some embodiments, at least one of the cancer cells (e.g., stromal cells) in the patient over-expresses CD73.

Non-limiting examples of cancers include bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary, adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the composition include but are not limited to intradermal, intramuscular, intraperitoneal, intravenous, and subcutaneous routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

The amount of the antibodies of the disclosure which will be effective in the treatment, inhibition and prevention of an inflammatory, immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

As a general proposition, the dosage administered to a patient of the antigen-binding polypeptides of the present disclosure is typically 0.1 mg/kg to 100 mg/kg of the patient's body weight, between 0.1 mg/kg and 20 mg/kg of the patient's body weight, or 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation.

The methods for treating an infectious or malignant disease, condition or disorder comprising administration of a composition of the disclosure are typically tested in vitro, and then in vivo in an acceptable animal model, for the desired therapeutic or prophylactic activity, prior to use in humans. Suitable animal models, including transgenic animals, are well known to those of ordinary skill in the art. For example, in vitro assays to demonstrate the therapeutic utility of antigen-binding polypeptide described herein include the effect of an antigen-binding polypeptide on a cell line or a patient tissue sample. The effect of the antigen-binding polypeptide on the cell line and/or tissue sample can be determined utilizing techniques known to those of skill in the art, such as the assays disclosed elsewhere herein. In accordance with the disclosure, in vitro assays which can be used to determine whether administration of a specific antigen-binding polypeptide is indicated, include in vitro cell culture assays in which a patient tissue sample is grown in culture, and exposed to or otherwise administered a compound, and the effect of such compound upon the tissue sample is observed.

In a further embodiment, the compositions of the disclosure are administered in combination with an antineoplastic agent, an antiviral agent, antibacterial or antibiotic agent or antifungal agents. Any of these agents known in the art may be administered in the compositions of the current disclosure.

In another embodiment, compositions of the disclosure are administered in cot bination with a chemotherapeutic agent. Chemotherapeutic agents that may be administered with the compositions of the disclosure include ; but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15 anti-CD40, CD40L, and TNF-α.

In additional embodiments, the compositions of the disclosure are administered in combination with other therapeutic or prophylactic regimens, such as, for example, radiation therapy.

### EXAMPLES

The disclosure is further understood by reference to the following examples, which are intended to be purely exemplary of the disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description and accompanying figures. Such modifications fall within the scope of the appended claims.

### Example 1, Development of Anti-CD73 Antibody IM005

A humanized anti-CD73 antibody (IM005) was developed that exhibited strong activities in binding to and inhibiting the enzymatic activity of the human CD73 protein. In addition, as demonstrated in WO2018137598, the antibody effectively induces the internalization of cell surface CD73. The antibody can also completely reverse AMP and tumor cell-mediated suppression of CD4⁺ and CD8⁺ T cell responses, and is efficacious in the suppression of tumor-derived CD73 activity, leading to inhibition of tumor growth.

Binding analysis has shown that IM005 binds to the C-terminal domains of the CD73 protein which differs from known anti-CD-73 antibodies that bind to the N-terminal domains. The unique binding property of IM005 contributes to its superior CD73 inhibition profile as compared to known antibodies. For instance, MEDI-9447 (Oleclumab) is a human anti-CD73 monoclonal antibody currently under clinical development for the treatment of pancreatic and colorectal and other cancers. Inhibition of CD73 by MEDI-9447 requires that both binding sites on a full MEDI-9447 antibody bind to CD73, while a monovalent binding by Hu101-28 is sufficient. Therefore, MEDI-9447 is incapable of inhibiting soluble CD73, or on cells hat express relatively low level of CD73. By contrast, IM005 can achieve complete inhibition of CD73 activity on cells which express different levels of CD73 on the surface, and soluble CD73.

IM005 has the following VH//VL and CDR sequences.

TABLE 1
Anti-CD73 CDRs

| | | SEQ ID |
|---|---|---|
| Name | Sequences | NO: |
| VH CDR1 | SGYYWN | 1 |
| VH CDR2 | YINYGGSNGYNPSLKS | 2 |
| VH CDR3 | DYDAYYEALDD | 3 |
| VL CDR1 | RASSRVNYMH | 4 |
| VL CDR2 | ATSNLAS | 5 |
| VL CDR3 | QQWSSNPPT | 6 |
| VH | EVQLQESGPGLVKPSETLSLTCAVSGYSITSGYYWNWIRQPPGKKLEWMG | 7 |
| | YINYGGSNGYNPSLKSRITISRDTSKNQFSLKLSSVTAADTAVYYCARDY | |
| | DAYYEALDDWGQGTTVTVSS | |
| VL | EIVLSQSPATLSLSPGERATLSCRASSRVN-YMHWYQQKPGQSPRPWISA | 8 |
| | TSNLASGVPARFSGSGSGTSYTLTISSLEPEDFAVYYCQQWSSNPPTFGG | |
| | GTKVEIK | |
| Heavy | EVQLQESGPGLVKPSETLSLTCAVSGYSITSGYYWNWIRQPPGKKLEWMG | 9 |
| Chain | YINYGGSNGYNPSLKSRITISRDTSKNQFSLKLSSVTAADTAVYYCARDY | |
| | DAYYEALDDWGQGTTVTVSS | |
| | ASTKGPSVFPLAPSSKSTSGGTAALGCLVKDYFPEPVTVSWNSGALTSGV | |
| | HTFPAVLQSSGLYSLSSVVTVPSSSLGTQTYICNVNHKPSNTKVDKKVEP | |
| | KSCDKTHTCPPCPAPELLGGPSVFLFPPKPKDTLMISRTPEVTCVVVDVS | |
| | HEDPEVKFNWYVDGVEVHNAKTKPREEQYASTYRVVSVLTVLHQDWLNGK | |
| | EYKCKVSNKALPAPIEKTISKAKGQPREPQVYTLPPSREEMTKNQVSLTC | |
| | LVKGFYPSDIAVEWESNGQPENNYKTTPPVLDSDGSFFLYSKLTVDKSRW | |
| | QQGNVFSCSVMHEALHNHYTQKSLSLSPGK | |
| Light | EIVLSQSPATLSLSPGERATLSCRASSRVNYMHWYQQKPGQSPRPWISAT | 10 |
| Chain | SNLASGVPARFSGSGSGTSYTLTISSLEPEDFAVYYCQQWSSNPPTFGGG | |
| | TKVEIK | |
| | RTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTK | |
| | SFNRGEC | |

To prepare the IM005 antibody, VH and VK genes were produced synthetically and then respectively cloned into vectors containing the human gamma 1 and human kappa constant domains.

### Example 2. Buffer and pH Selection

This example screened for optimal pH/buffer condition that stabilized the protein most and to study the protein solubility that will support subsequent formulation development of IM005.

### Methods

### Protein Concentration

Protein concentration was determined by a Thermo LTV spectrophotometer. The extinction coefficient used in pH and buffer evaluation study was 1,660 AU*mL*mg⁻¹*cm⁻, which was applied in all fbrmulation development studies. All measurements were repeated twice with 2.5 μL sample each time and an average was taken.

### SEC-HPLC

Size exclusion chromatography was performed using an Agilent HPLC system with a TSKGel G3000SWXL column (300×7.8 mm, 5 μm). The mobile phase was 50 mM PB, 300 mM NaCl, pH 6.8±0.2 and the flow rate was set as 1.0 mL/min. 100 ug protein for each sample was injected and the wavelength of detector was set at 280 nm.

cIEF

cIEF was performed on a ProteinSitnple iCE analyzer equipped with FC-coated cIEF cartridge. The cIEF method was used to analyze isoelectric point (pI) and charge variants of IM005 samples. The loading mixture contained 20 μL sample at a concentration of 1.0 mg/mL, 35 μL 1% Methyl cellulose, 1 μL Pharmalyte 3-10 and 3 μL Pharmalyte 8-10.5 carrier ampholyte, 0.5 μL low pI Marker 6.61 or 6.14, 0.5 μL high pI Marker 9.46 or 9.77, 25 μL 8 mol/L urea, and 15 μL purified water to make the loading sample volume to 100 μL finally. Temperature of auto-sampler was 5° C., sample injection duration was 90 seconds. Focusing was performed in two stages: prefocusing for 1 min under 1500 V, followed by focusing at 3000 V for 8 min. Signals were detected at 280 nm with whole column imaging detection technology. Data were analyzed using Chromperfect Analysis Software.

### Turbidity (UV350)

Turbidity was performed by a spectrophotometer (Spectra Max). 150 μL samples were added into the wells of a 96-well plate, 150 μL of respective buffers were also added into the corresponding wells as the reference. Then the absorption of the buffers and samples were tested at 350 nm. The UV350 value of the protein was obtained by subtracting the corresponding buffer.

### Caliper-SDS (Reduced & Non-Reduced)

Caliper-SDS was performed by Caliper- a microchip-based assay. Non-reduced: The denaturing solution was prepared by mixing sample buffer (from kit), 10% SDS and 100 mM Ethylmaleimide at 20:1:0.7 volume ratio. Two microliters of samples and 7 μL denaturing solution were mixed well, incubated at 70° C. for 10 mins and spun down. H₂O (35 μL) was added to the sample, then 42 μL of the mixture was transferred into 96-well plate and centrifuged at 4000 rpm for 20 mins to remove air bubbles. After the plate was loaded onto the instrument's plate holder, samples were sipped, stained, separated and detected in the Microchip which was filled with destaining-gel, fluorescent dye and marker. Data was analyzed with LabChip GX Reviewer.

Reduced: The denaturing solution was prepared by mixing sample buffer (from kit), 10% SDS and 1 M Dithiothreitol at 20:1:0.7 volume ratio, while the reference standard or samples were diluted to 1 mg/mL with MQ WO. Two microliters of samples and 7 μL denaturing solution were mixed well, incubated at 70° C. for 10 mins and spun down.

H₂O (35 μL) was added to the sample, then 42 μL of the mixture was transferred into 96-well plate and centrifuged at 4000 rpm for 2.0 mins to remove air bubbles. Afterthe plate was loaded onto the instrument's plate holder, samples were sipped, stained, separated and detected in the Microchip which was filled with de-staining-gel, fluorescent dye and marker. Data was analyzed with LabChip GX Reviewer.

### DSC

DSC analysis was performed with a GE DSC System. Samples were diluted to 1 mg/mL with reference buffer. 400 μL of respective reference buffers were added into the odd-numbered wells of a 96-well plate and 400 μL of samples were added into the even-numbered wells of the same plate. Experimental parameters were set such that the scan temperature ramped from 10-110° C. with a rate of 200° C./h. Analysis of thermograms was performed with MicroCal VP-Capillary DSC software.

### Viscosity

Viscosity was assessed using a BROOKFIELD viscometer and the cone type was CPA-40Z. The test temperature was 25° C. and 0.5 mL sample was required.

### Results

IM005 (20.4 mg/mL) was first exchanged into 9 different buffers as shown in Table 1. The protein concentration was adjusted to 10 mg/mL.

TABLE 1
Buffer Conditions

| | Protein | | |
|---|---|---|---|
| No. | Concentration | Buffer | pH |
| F1 | 10 mg/mL | 20 mM acetate | 4.5 |
| F2 | | 20 mM acetate | 5.0 |
| F3 | | 20 mM acetate | 5.5 |
| F4 | | 20 mM histidine | 5.5 |
| F5 | | 20 mM histidine | 6.0 |
| F6 | | 20 mM histidine | 6.5 |
| F7 | | 20 mM PB | 6.0 |
| F8 | | 20 mM PB | 6.5 |
| F9 | | 20 mM PB | 7.0 |

Four 2 mL glass vials were filled with 1 mL filtered product for each formulation. The vials were stoppered and crimped immediately after filling. Apart from crimping, all procedures were carried out in a bio-safety hood. The vials were placed into 40° C. incubator, and vials were removed from 40° C. condition for analysis at different time points.

### DSC

The thermodynamic stability of each formulation was examined using DSC. The melting temperature (Tm) of heat induced unfolding of protein is considered an indicator of its conformational stability.

As shown in Table 2, the onset temperatures of transition ranged from 46.18° C. (F1: Acetate pH4.5) to 57.25° C. (F7: PB pH6.0). The Tm1 ranged from 54.53° C. (F1 Acetate pH4.5) to 7657° C. (F3: Acetate pH5.5), and Tm2 range from 77.27° C. (F5: Histidine pH6.0) to 86.54° C. (F9: PB pH7.0) (except F3: Acetate pH5.5, F6: Histidine pH6.5 and F7: PB pH6.0). The different temperature of transitions were observed among the profiles of 9 formulations.

TABLE 2
DSC Result of pH/buffer Screening Study

| | TM Onset | Tm1 | Tm2 |
|---|---|---|---|
| pH/Buffer | (° C.) | (° C.) | (° C.) |
| F1: 20 mM Acetate, pH 4.5 | 46.18 | 54.53 | 78.43 |
| F2: 20 mM Acetate, pH 5.0 | 49.15 | 60.27 | 78.05 |
| F3: 20 mM Acetate, pH 4.5 | 56.53 | 76.57 | NA |
| F4: 20 mM Histidine, pH 5.5 | 49.58 | 59.61 | 77.96 |
| F5: 20 mM Histidine, pH 4.5 | 52.80 | 63.36 | 77.27 |
| F6: 20 mM Histidine, pH 4.5 | 56.33 | 75.78 | NA |
| F7: 20 mM PB, pH 6.0 | 57.25 | 74.47 | NA |
| F8: 20 mM PB, pH 6.5 | 56.80 | 72.37 | 86.26 |
| F9: 20 mM PB, pH 7.0 | 57.05 | 69.84 | 86.54 |

### Appearance

Appearance evaluation results are shown in Table 3. At 40° C. conditions, visible particles were observed in F6 (Histidine pH6.5), F7 (PB pH6.0), F8 (PB pH6.5) and F9 (PB pH 7.0) after 2 weeks. And visible particles were observed in F3 (Acetate pH5.5), F6 (Histidine pH6.5), F7 (PB pH6.0), F8 (PB p116.5) and F9 (PB pH7.0) after 4 weeks. No visible particles were observed in F1 (Acetate pH4.5), F2 (Acetate pH5.0), F4 (Histidine pH5.5) and F5 (Histidine pH6.0) after incubation at 40° C.

pH

The pH values of all formulations were measured at all sampling points. Results showed that there was no substantial change in pH value after incubation at 40° C.

### Protein Concentration

Protein concentrations of all formulations were measured at all sampling points, using nanodrop2000, a UV280 method. As the result shows, about 10% decrease in protein concentration was observed in F9 (PB pH7.0) and no substantial change among F1-F8 in protein concentration was seen after incubation at 40° C. condition.

TABLE 3
Appearance, pH, UV280 Result of pH/buffer Screening: 40° C.

| | | | | UV280 |
|---|---|---|---|---|
| 40° C. | | Appearance | pH | (mg/mL) |
| F1: 20 mM | T0 | Clear, light yellow, free of particle | 4.5 | 10.2 |
| Acetate, pH | 1 W | Clear, light yellow, free of particle | 4.6 | 10.2 |
| 4.5 | 2 W | Clear, light yellow, free of particle | 4.6 | 10.2 |
| | 4 W | Clear, light yellow, free of particle | 4.6 | 10.4 |
| F2: 20 mM | T0 | Clear, light yellow, free of particle | 5.0 | 10.2 |
| Acetate, pH | 1 W | Clear, light yellow, free of particle | 5.1 | 10.2 |
| 5.0 | 2 W | Clear, light yellow, free of particle | 5.1 | 10.3 |
| | 4 W | Clear, light yellow, free of particle | 5.1 | 10.3 |
| F3: 20 mM | T0 | Clear, light yellow, free of particle | 5.5 | 10.1 |
| Acetate, pH | 1 W | Clear, light yellow, free of particle | 5.6 | 10.0 |
| 5.5 | 2 W | Clear, light yellow, free of particle | 5.6 | 10.1 |
| | 4 W | A few of particles | 5.6 | 10.0 |
| F4: 20 mM | T0 | Clear, light yellow, free of particle | 5.6 | 10.2 |
| Histidine, | 1 W | Clear, light yellow, free of particle | 5.5 | 10.1 |
| pH 5.5. | 2 W | Clear, light yellow, free of particle | 5.5 | 10.2 |
| | 4 W | Clear, light yellow, free of particle | 5.6 | 10.2 |
| F5: 20 mM | T0 | Clear, light yellow, free of particle | 6.2 | 10.0 |
| Histidine, | 1 W | Clear, light yellow, free of particle | 6.1 | 10.1 |
| pH 6.0 | 2 W | Clear, light yellow, free of particle | 6.1 | 10.1 |
| | 4 W | Clear, light yellow, free of particle | 6.1 | 10.2 |
| F6: 20 mM | T0 | Clear, light yellow, free of particle | 6.6 | 10.2 |
| Histidine, | 1 W | Clear, light yellow, free of particle | 6.5 | 10.1 |
| pH 6.5 | 2 W | A few of particles | 6.5 | 10.2 |
| | 4 W | A few of particles | 6.5 | 10.2 |
| F7: 20 mM | T0 | Clear, light yellow, free of particle | 6.0 | 10.3 |
| PB, pH 6.0 | 1 W | Clear, light yellow, free of particle | 6.1 | 10.3 |
| | 2 W | A few of particles | 6.1 | 10.4 |
| | 4 W | A few of particles | 6.1 | 10.4 |
| F8: 20 mM | T0 | Clear, light yellow, free of particle | 6.5 | 10.5 |
| PB, pH 6.5 | 1 W | Clear, light yellow, free of particle | 6.5 | 10.5 |
| | 2 W | A few of particles | 6.5 | 10.4 |
| | 4 W | A few of particles | 6.5 | 10.3 |
| F9: 20 mM | T0 | Clear, light yellow, free of particle | 7.0 | 9.8 |
| PB, pH 7.0 | 1 W | Clear, light yellow, free of particle | 7.0 | 9.8 |
| | 2 W | A few of particles | 7.0 | 9.6 |
| | 4 W | Many particles | 6.9 | 8.9 |

### SEC-HPLC

SEC results at 40° C. are shown in Table 4. Aggregation was observed in all formulations and fragmentation was observed in some buffers. % Main peak from all 9 formulations reduced with increasing thermal treatment time. The more substantial reductions were observed in F1 (Acetate pH4.5), F7 (PB pH6.0), F8 (PB pH6.5), and F9 (PB pH7.0) samples. These results are consistent with % LMW and % HMW species.

Based on the SEC-HPLC results, F2 to F6 can be selected as formulation candidates.

TABLE 4
SEC-HPLC Result of pH/buffer Screening: 40° C.

| | | SEC_HPLC | | |
|---|---|---|---|---|
| | | | % | |
| | | % | Main | % |
| 40° C. | | HMW | peak | LMW |
| F1: 20 mM Acetate, pH 4.5 | T0 | 0.6 | 99.4 | NA |
| | 1 W | 1.3 | 98.2 | 0.5 |
| | 2 W | 1.5 | 97.7 | 0.8 |
| | 4 W | 2.5 | 96.4 | 1.1 |
| F2: 20 mM Acetate, pH 5.0 | T0 | 0.4 | 99.6 | NA |
| | 1 W | 0.5 | 99.1 | 0.4 |
| | 2 W | 0.6 | 98.9 | 0.5 |
| | 4 W | 1.0 | 98.4 | 0.6 |
| F3: 20 mM Acetate, pH 5.5 | T0 | 0.9 | 99.1 | NA |
| | 1 W | 1.2 | 98.5 | 0.3 |
| | 2 W | 1.3 | 98.4 | 0.3 |
| | 4 W | 1.6 | 97.8 | 0.5 |
| F4: 20 mM Histidine, pH 5.5 | T0 | 0.7 | 99.3 | NA |
| | 1 W | 0.8 | 98.8 | 0.3 |
| | 2 W | 0.9 | 98.7 | 0.4 |
| | 4 W | 1.2 | 98.2 | 0.6 |
| F5: 20 mM Histidine, pH 6.0 | T0 | 0.4 | 99.6 | NA |
| | 1 W | 0.6 | 99.1 | 0.3 |
| | 2 W | 0.6 | 99.1 | 0.3 |
| | 4 W | 0.9 | 98.6 | 0.5 |
| F6: 20 mM Histidine, pH 6.5 | T0 | 0.9 | 99.1 | NA |
| | 1 W | 1.1 | 98.6 | 0.3 |
| | 2 W | 1.2 | 98.3 | 0.5 |
| | 4 W | 1.6 | 97.7 | 0.6 |
| F7: 20 mM PB, pH 6.0 | T0 | 0.9 | 99.1 | NA |
| | 1 W | 1.6 | 98.1 | 0.3 |
| | 2 W | 1.9 | 97.8 | 0.4 |
| | 4 W | 2.6 | 96.6 | 0.8 |
| F8: 20 mM PB, pH 6.5 | T0 | 0.7 | 99.3 | NA |
| | 1 W | 1.8 | 98.0 | 0.2 |
| | 2 W | 2.2 | 97.4 | 0.4 |
| | 4 W | 3.2 | 96.0 | 0.7 |
| F9: 20 mM PB, pH 7.0 | T0 | 1.4 | 98.6 | NA |
| | 1 W | 2.2 | 97.6 | 0.2 |
| | 2 W | 2.7 | 96.9 | 0.4 |
| | 4 W | 3.2 | 96.1 | 0.7 |

### Caliper-SDS (Reduced and Non-Reduced)

Caliper-SDS is a CE based high-throughput analysis, which is capable of detection of fragmentation of monoclonal antibodies with high sensitivity. In Caliper-SDS experiments, more substantial % purity reductions were observed in F1 (Acetate pH4.5), F7 (PB pH6.0), F8 (PB pH6.5), and F9 (PB pH7.0) non-reduced samples in Table 5. In addition, % (LC+HC) reduction were more rapid in F1, F7, F8, and. F9 samples. These results are consistent with the SEC results.

TABLE 5
Caliper-SDS (Reduced and Non-Reduced)
Result of pH/buffer Screening (40° C.)

| | | rCaliper-SDS | NrCaliper-SDS |
|---|---|---|---|
| 40° C. | | % LC + % HC | % Main Peak |
| F1: 20 mM Acetate, pH 4.5 | T0 | 99.9 | 98.4 |
| | 1 W | 99.6 | 97.0 |
| | 2 W | 99.0 | 95.7 |
| | 4 W | 97.7 | 93.2 |
| F2: 20 mM Acetate, pH 5.0 | T0 | 99.9 | 98.3 |
| | 1 W | 99.8 | 97.3 |
| | 2 W | 99.5 | 96.5 |
| | 4 W | 99.0 | 94.9 |
| F3: 20 mM Acetate, pH 5.5 | T0 | 99.9 | 98.3 |
| | 1 W | 99.9 | 97.8 |
| | 2 W | 99.6 | 96.8 |
| | 4 W | 99.2 | 95.2 |
| F4: 20 mM Histidine, pH 5.5 | T0 | 99.9 | 98.2 |
| | 1 W | 99.8 | 97.6 |
| | 2 W | 99.5 | 96.8 |
| | 4 W | 99.1 | 95.5 |
| F5: 20 mM Histidine, pH 6.0 | T0 | 99.9 | 98.3 |
| | 1 W | 99.9 | 97.6 |
| | 2 W | 99.5 | 96.9 |
| | 4 W | 99.2 | 95.6 |
| F6: 20 mM Histidine, pH 6.5 | T0 | 99.9 | 98.3 |
| | 1 W | 99.7 | 97.7 |
| | 2 W | 99.4 | 96.8 |
| | 4 W | 99.2 | 95.1 |
| F7: 20 mM PB, pH 6.0 | T0 | 99.9 | 98.3 |
| | 1 W | 99.7 | 97.1 |
| | 2 W | 99.3 | 96.4 |
| | 4 W | 98.9 | 94.1 |
| F8: 20 mM PB, pH 6.5 | T0 | 99.9 | 98.1 |
| | 1 W | 99.6 | 97.5 |
| | 2 W | 99.1 | 95.9 |
| | 4 W | 98.7 | 92.7 |
| F9: 20 mM PB, pH 7.0 | T0 | 99.9 | 98.4 |
| | 1 W | 99.4 | 96.1 |
| | 2 W | 98.9 | 95.3 |
| | 4 W | 98.3 | 91.7 |

Caliper-SDS result shown the similar trend as other assay results that purity decreased more in low pH (pH4.5) and high pH (pH7.0) conditions.

cIEF

After 4 weeks incubation at 40° C., cIEF data were obtained and are presented in Table 6, The results shown the similar trend as SEC-HPLC, % main peak reductions were more substantial in F1 (Acetate pH4.5), F7 (PB pH6.0), F8 (PB pH6.5), and F9 (PB pH7.0) samples. In each buffer type, % basic peak was reduced with pH increasing but % acidic peak was increased with pH increasing.

TABLE 6
cIEF Result of pH/buffer Screening: 40° C.

| | | CIEF | | |
|---|---|---|---|---|
| | | % | % | % |
| | | Acidic | Main | Basic |
| 40° C. | | Peak | Peak | Peak |
| F1: 20 mM | T0 | 23.0 | 55.8 | 21.2 |
| Acetate, pH 4.5 | 1 W | 22.6 | 47.8 | 29.6 |
| | 2 W | 25.3 | 38.1 | 36.6 |
| | 4 W | 26.2 | 33.3 | 40.5 |
| F2: 20 mM | T0 | 23.4 | 54.4 | 22.2 |
| Acetate, pH 5.0 | 1 W | 20.6 | 52.6 | 26.8 |
| | 2 W | 26.9 | 43.2 | 29.9 |
| | 4 W | 30.2 | 38.0 | 31.8 |
| F3: 20 mM | T0 | 19.5 | 58.6 | 21.9 |
| Acetate, pH 5.5 | 1 W | 24.1 | 52.3 | 23.5 |
| | 2 W | 29.2 | 46.0 | 24.8 |
| | 4 W | 32.9 | 41.0 | 26.1 |
| F4: 20 mM | T0 | 23.1 | 55.9 | 20.9 |
| Histidine, pH | 1 W | 21.6 | 54.4 | 24.0 |
| 5.5 | 2 W | 27.2 | 44.5 | 28.3 |
| | 4 W | 30.5 | 40.2 | 29.3 |
| F5: 20 mM | T0 | 22.9 | 56.1 | 21.0 |
| Histidine, pH | 1 W | 23.2 | 54.2 | 22.5 |
| 6.0 | 2 W | 28.3 | 47.2 | 24.5 |
| | 4 W | 34.1 | 42.4 | 23.5 |
| F6: 20 mM | T0 | 22.5 | 56.8 | 20.7 |
| Histidine, pH | 1 W | 25.5 | 54.5 | 20.0 |
| 6.5 | 2 W | 30.2 | 48.1 | 21.7 |
| | 4 W | 39.6 | 41.1 | 19.2 |
| F7: 20 mM PB, | T0 | 22.1 | 57.3 | 20.6 |
| pH 6.0 | 1 W | 25.4 | 53.8 | 20.8 |
| | 2 W | 33.1 | 44.1 | 22.8 |
| | 4 W | 41.5 | 38.3 | 20.2 |
| F8: 20 mM PB, | T0 | 21.8 | 56.1 | 22.0 |
| pH 6.5 | 1 W | 28.0 | 52.1 | 19.9 |
| | 2 W | 38.6 | 42.9 | 18.5 |
| | 4 W | 49.7 | 34.3 | 16.0 |
| F9: 20 mM PB, | T0 | 22.3 | 56.6 | 21.2 |
| pH 7.0 | 1 W | 34.1 | 47.6 | 18.3 |
| | 2 W | 45.5 | 37.7 | 16.8 |
| | 4 W | 59.1 | 28.8 | 12.1 |

These results show the most charge variants change was observed in F9 (PB pH7.0) and the least charge variants change was observed in F5 (Histidine pH6.0).

In summary, in the pH/buffer screening study, visible particles were observed in F3 (acetate pH5.5), F6 (histidine pH6.5), F7 (PB pH6.0), F8 (PB pH6.5) and F9 (PB pH7.0) after incubation at 40° C. At 40° C., F1 (Acetate, pH4.5) and F7, F8, F9 (Phosphate buffers) not only showed the most notable purity decrease in Caliper-SDS (reduced and non-reduced) and. SEC result, but also witnessed the most charge variants change in cIEF. And 10% decrease of concentration was observed in F9 after incubation at 40° C.

These results, therefore, indicate that IMOOS was less stable in phosphate buffer and the protein might be unstable when buffer pH is too low. For F2, F4 and F5, no substantial change was observed in Caliper-SDS reduced results after 4 weeks incubation at 40° C., while decrease in % purity of Caliper-SDS non-reduced was observed. F5 decrease less than others in both Caliper-SDS non-reduced and. SEC purity results. Therefore, F5 (20mM histidine pH 6.0 buffer) was selected for further development.

### Example 3. Solubility Study

This example evaluated the solubility of IM005 in the selected buffering system.

IM005 was exchanged into 20 mM histidine, pH 6.0 buffer and the concentration was adjusted to 80 mg/mL and 150 mg/mL. The samples were aseptically filtered with 0.22 μm PVDF membrane filter. For each concentration, 3 2-mL glass vials were filled with 1 mL of DS in a bio-safety hood. The vials were stoppered and crimped immediately after filling.

A total of 6 vials were placed into different incubators (2-8° C. and 25° C.), and 1 vial was removed from each condition for analysis.

Appearance, concentration (UV280) and turbidity (UV350) were measured at all sampling points (results in Table 7). The results showed that there was no substantial change after incubation at 2-8° C. and 25° C. for 48 hours.

TABLE 7
Appearance, Concentration and Turbidity Result of Solubility Study: 2-8° C. and 25° C.

| pH/buffer | Protein | Time point | Appearance | UV280 (mg/ml) | Turbidity | Viscosity (cP) |
|---|---|---|---|---|---|---|
| 20 mM | 80 mg/mL | T0 | Clear, light | 80 | 0.071 | NA |
| histidine, | | 2-8° C. 48 H | yellow, | 80 | 0.075 | |
| pH6.0 | | 25° C. 48 H | free of | 80 | 0.068 | |
| | 150 mg/mL | T0 | particle | 146 | 0.120 | 24.5 |
| | | 2-8° C. 48 H | | 146 | 0.110 | |
| | | 25° C. 48 H | | 148 | 0.093 | |

IM005 was successfully concentrated to the target concentrations of 80 mg/mL and 150 mg/mL in 20 mM histidine, pH6.0 buffer. The short-term stability results showed that no substantial change was observed after storage at 2˜8° C. and 25° C. for 48 hours. It indicated that IM005 was stable after short term storage at 80 mg/mL or 150 mg/mL in 20 mM histidine, pH 6.0 buffer.

The pH/buffer screening study investigated 9 pH/buffer types under stress condition (40° C.), and the result showed stability of IM005 is closely related with both pH and buffer type. Poor protein stability was observed under low pH, high pH conditions, and phosphate buffers. The selected buffer was 20 mM histidine, pH6.0 buffer (F5).

In solubility study, no obvious change was observed at 2˜8° C. and 25° C. for 48 hours, indicating that the IM005 was stable in 80 mg/mL and 150 mg/mL concentration for short term.

Based on results from the pH/buffer screening study and solubility study, 20 mM histidine, pH6.0 buffer will be used in the following formulation development study for the product.

### Example 4. Screening of Excipients

This example examined candidates excipients that could stabilize the IM005 protein in a 20 mM histidine, pH 6.0 buffer system.

IM005 antibody stock solutions were exchanged into 20 mM histidine pH 6.0 buffer system. Then, the excipients listed in Table 8 were added to this solution, respectively. The protein concentration was adjusted to 50 mg/mL.

TABLE 8
Candidates of Formulation Recipes

| | Protein | | | | |
|---|---|---|---|---|---|
| No. | Concentration | Buffer | Excipient | Surfactant | pH |
| F1 | 50 mg/mL | 20 mM | 80 g/L Sucrose | 0.02% PS | 6.0 |
| F2 | | Histidine | 80 g/L Trehalose | 80 | |
| | | | dihydrate | | |
| F3 | | | 40 g/L Sorbitol | | |
| F4 | | | 40 g/L Mannitol | | |
| F5 | | | 140 mM Arginine | | |
| | | | hydrochloride | | |
| F6 | | | 80 g/L Sucrose | NA | |

All formulation samples were eventually filtered with 0.22 μm PVDF membrane filter, filled into 2 mL, glass vials (1 mL/vial), stoppered and sealed in bio-safety hood. The vials were placed into 25° C., 40° C. incubator and −40° C. refrigerator apart from T0 samples, and vials were removed from these conditions for analysis.

Advanced 2020 Multi-Sample osmometer was used for osmolality measurement of 6 formulations. The results are shown in Table 9.

TABLE 9
Osmolality Result of Excipient Screening Study: T0

| | | Osmolality |
|---|---|---|
| No. | Formulation Composition | (mOsmol/kg) |
| F1 | 20 mM Histidine, 8% Sucrose, | 334 |
| | 0.02% PS80, pH 6.0 | |
| F2 | 20 mM Histidine, 8% Trehalose | 327 |
| | dihydrate, 0.02% PS80, pH 6.0 | |
| F3 | 20 mM Histidine, 4% Sorbitol, | 327 |
| | 0.02% PS80, pH 6.0 | |
| F4 | 20 mM Histidine, 4% Mannitol, | 303 |
| | 0.02% PS80, pH 6.0 | |
| F5 | 20 mM Histidine, 140 mM | 344 |
| | Arginine•HCl, 0:02% PS80, | |
| | pH 6.0 | |
| F6 | 20 mM Histidine, 8% sucrose, | 332 |
| | pH 6.0 | |

### DSC

The thermodynamic stability of each formulation was examined using DSC. The melting temperature (Tm) induces the temperature of start of unfolding of protein is considered as an indicator of its conformational stability.

As shown in Table 10, the onset temperatures of transition ranged from 50.9° C. (F5: Arginine·HCl, pH 6.0) to 58.6° C. (F2: Trehalose, pH 6.0). The Tm1 ranged from 59.3° C. (F5: Arginine·HCl, pH 6.0) to 65.2° C. (F1: Sucrose, pH 6.0), and Tm2 range from 76.7° C. (F5: Arginine·HCl, pH 6.0) to 78.5° C. (F1: Sucrose, pH 6.0). The maximum difference values of Tmₒₙₛₑₜ, Tm1 and Tm2 among 6 formulations are 7.7° C., 5.8° C. and 1.9° C., respectively.

TABLE 10
DSC Result of Excipient Screening Study

| | | Tmₒₙₛₑₜ | Tm1 | Tm2 |
|---|---|---|---|---|
| No. | Formulation Composition | (° C.) | (° C.) | (° C.) |
| F1 | 20 mM Histidine, 8% Sucrose, | 56.2 | 65.2 | 78.5 |
| | 0.02% PS80, pH 6.0 | | | |
| F2 | 20 mM Histidine, 8% Trebalose | 58.6 | 64.7 | 78.1 |
| | dihydrate, 0.02% PS80, pH 6.0 | | | |
| F3 | 20 mM Histidine, 4% Sorbitol, | 57.5 | 64.2 | 78.1 |
| | 0.02% PS80, pH 6.0 | | | |
| F4 | 20 mM Histidine, 4% Mannitol, | 58.3 | 63.9 | 77.8 |
| | 0.02% PS80, pH 6.0 | | | |
| F5 | 20 mM Histidine, 140 mM | 50.9 | 59.3 | 76.7 |
| | Arginine•HCl, 0.02% PS80, | | | |
| | pH 6.0 | | | |
| F6 | 20 mM Histidine, 8% Sucrose, | 58.2 | 65.0 | 78.3 |
| | pH 6.0 | | | |

### Appearance

As showed in Tables 11-13, after incubation at 40° C. for 2 and 4 weeks, no visible particles were observed in all formulations except F6 (without PS80). While after incubation at 25° C. for 4 weeks, no visible particles were observed in all 6 formulations. Under the freeze-thaw (−40° C. to RI) conditions, visible particles were observed in F4 (mannitol, pH 6.0) after 10 cycles of freeze-thaw, but not in other 5 formulations.

pH

The pH values of 6 formulations were measured at all sampling points as shown in Tables 11-13. Results showed that there was no substantial change in pH value after incubation at 25° C. 40° C. for 4 weeks or repeated freeze-thaw for 5 cycles/10 cycles.

### Protein Concentration (A280)

Protein concentrations of 6 formulations were measured at all sampling points as shown in Tables 11-13, using nanodrop2000, a UV280 method. As the results showed, there was no substantial change in protein concentration after incubation t 25° C., 40° C. for 4 weeks or repeated freeze-thaw for 5 cycles 10 cycles.

TABLE 11
Appearance, pH, UV280 Result of
Excipient Screening Study: 40° C.

| | | | | UV280 |
|---|---|---|---|---|
| 40° C. | | Appearance | pH | (mg/mL) |
| F1: 20 mM Histidine, | T0 | Clear, light yellow, | 6.0 | 50.6 |
| 8% Sucrose, 0.02% | | free of particle | | |
| PS80, pH 6.0 | 2 W | Clear, light yellow, | 6.1 | 51.1 |
| | | free of particle | | |
| | 4 W | Clear, light yellow, | 6.2 | 52.7 |
| | | free of particle | | |
| F2: 20 mM Histidine, | T0 | Clear, light yellow, | 6.1 | 52.0 |
| 8% Trehalose dihydrate, | | free of particle | | |
| 0.02% PS80, pH 6.0 | 2 W | Clear, light yellow, | 6.1 | 51.8 |
| | | free of particle | | |
| | 4 W | Clear, light yellow, | 6.2 | 52.9 |
| | | free of particle | | |
| F3: 20 mM Histidine, | T0 | Clear, light yellow, | 6.1 | 50.9 |
| 4% Sorbitol, 0.02% | | free of particle | | |
| PS80, pH 6.0 | 2 W | Clear, light yellow, | 6.1 | 50.6 |
| | | free of particle | | |
| | 4 W | Clear, light yellow, | 6.2 | 51.5 |
| | | free of particle | | |
| F4: 20 mM Histidine, | T0 | Clear, light yellow, | 6.1 | 51.5 |
| 4% Mannitol, 0.02% | | free of particle | | |
| PS80, pH 6.0 | 2 W | Clear, light yellow, | 6.1 | 49.9 |
| | | free of particle | | |
| | 4 W | Clear, light yellow, | 6.2 | 50.7 |
| | | free of particle | | |
| F5: 20 mM Histidine, | T0 | Clear, light yellow, | 6.1 | 50.5 |
| 140 mM Arginine•HCl, | | free of particle | | |
| 0.02% PS80, pH 6.0 | 2 W | Clear, light yellow, | 6.1 | 50.6 |
| | | free of particle | | |
| | 4 W | Clear, light yellow, | 6.0 | 51.7 |
| | | free of particle | | |
| F6: 20 mM Histidine, | T0 | Clear, light yellow, | 6.1 | 50.8 |
| 8% Sucrose, pH 6.0 | | free of particle | | |
| | 2 W | White particles | 6.1 | 50.9 |
| | 4 W | White particles | 6.1 | 51.5 |

TABLE 12
Appearance, pH, UV280 Result of
Excipient Screening Study: 25° C.

| | | | | UV280 |
|---|---|---|---|---|
| 25° C. | | Appearance | pH | (mg/mL) |
| F1: 20 mM Histidine, | T0 | Clear, light yellow, | 6.0 | 50.6 |
| 8% sucrose, 0.02% | | free of particle | | |
| PS80, H 6.0 | 4 W | Clear, light yellow, | 6.1 | 51.9 |
| | | free of particle | | |
| F2: 20 mM Histidine, | T0 | Clear, light yellow, | 6.1 | 52.0 |
| 8% Trehalose dihydrate, | | free of particle | | |
| 0.02% PS80, pH 6.0 | 4 W | Clear, light yellow, | 6.2 | 52.6 |
| | | free of particle | | |
| F3: 20 mM Histidine, | T0 | Clear, light yellow, | 6.1 | 50.9 |
| 4% Sorbitol, 0.02% | | free of particle | | |
| PS80, pH 6.0 | 4 W | Clear, light yellow, | 6.2. | 51.6 |
| | | free of particle | | |
| F4: 20 mM Histidine, | T0 | Clear, light yellow, | 6.1 | 51.5 |
| 4% Mannitol, 0.02% | | free of particle | | |
| PS80, pH 6.0 | 4 W | Clear, light yellow, | 6.1 | 50.1 |
| | | free of particle | | |
| F5: 20 mM Histidine, | T0 | Clear, light yellow, | 6.1 | 50.5 |
| 140 mM Arginine•HCl, | | free of particle | | |
| 0.02% PS80, pH 6.0 | 4 W | Clear, light yellow, | 6.1 | 51.8 |
| | | free of particle | | |
| F6: 20 mM Histidine, | T0 | Clear, light yellow, | 6.1 | 50.8 |
| 8% Sucrose, pH 6.0 | | free of particle | | |
| | 4 W | Clear, light yellow, | 6.1 | 51.6 |
| | | free of particle | | |

TABLE 13
Appearance, pH, UV280 Result of Excipient
Screening Study: Freeze thaw

| | | | | UV280 |
|---|---|---|---|---|
| Freeze thaw: −40° C. to RT | | Appearance | pH | (mg/mL) |
| F1: 20 mM Histidine, | T0 | Clear, light yellow, | 6.0 | 50.6 |
| 8% Sucrose, 0.02% | | free of particle | | |
| PS80, pH 6.0 | 5 FT | Clear, light yellow, | 6.1 | 51.5 |
| | | free of particle | | |
| | 10 FT | Clear, light yellow, | 6.1 | 52.3 |
| | | free of particle | | |
| F2: 20 mM Histidine, | T0 | Clear, light yellow, | 6.1 | 52.0 |
| 8% Trehalose dihydrate, | | free of particle | | |
| 0.02% PS80, pH 6.0 | 5 FT | Clear, light yellow, | 6.1 | 51.9 |
| | | free of particle | | |
| | 10 FT | Clear, light yellow, | 6.1 | 52.3 |
| | | free of particle | | |
| F3: 20 mM Histidine, | T0 | Clear, light yellow, | 6.1 | 50.9 |
| 4% Sorbitol, 0.02% | | free of particle | | |
| PS80, pH 6.0 | 5 FT | Clear, light yellow, | 6.1 | 50.6 |
| | | free of particle | | |
| | 10 FT | Clear, light yellow, | 6.1 | 51.3 |
| | | free of particle | | |
| F4: 20 mM Histidine, | T0 | Clear, light yellow, | 6.1 | S1.5 |
| 4% Mannitol, 0.02% | | free of particle | | |
| PS80, pH 6.0 | 5 FT | Clear, light yellow, | 6.1 | 49.5 |
| | | free of particle | | |
| | 10 FT | White particles | 6.1 | 49.5 |
| FS: 20 mM Histidine, | T0 | Clear, light yellow, | 6.1 | 50.5 |
| 140 mM Arginine•HCl, | | free of particle | | |
| 0.02% PS80, pH 6.0 | 5 FT | Clear, light yellow, | 6.1 | 51.5 |
| | | free of particle | | |
| | 10 FT | Clear, tight yellow, | 6.1 | 51.4 |
| | | free of particle | | |
| F6: 20 mM Histidine, | T0 | Clear, light yellow, | 6.1 | 50.8 |
| 8% Sucrose, pH 6.0 | | free of particle | | |
| | 5 FT | Clear, light yellow, | 6.1 | 51.3 |
| | | free of particle | | |
| | 10 FT | Clear, light yellow, | 6.1 | 51.9 |
| | | free of particle | | |

### SEC-HPLC

SEC-HPLC results at 40OC are shown in Table 14. From Fl to F6, % Main peak was decreased by 3.3%, 3.2%, 3.4%, 2.8%, 2.7% and 2.2%, respectively. And % HMW was increased by 2.3%, 2.2%, 2.5%, 1.8%, 1.7% and 1.3%, respectively, Meanwhile, % LMW was increased by 1.0%. (3.9%, 0 1.0%, 1.0% and 1.0%, respectively.

TABLE 14
SEC-HPLC Result of Excipient Screening Study: 40° C.

| | | SEC-HPLC | | |
|---|---|---|---|---|
| | | | % | |
| | | % | Main | % |
| 40° C. | | HMW | peak | LMW |
| F1: 20 mM Histidine, 8% Sucrose, | T0 | 0.6 | 99.4 | NA |
| 0.02% PS80, pH 6.0 | 40-2 W | 2.2 | 97.3 | 0.5 |
| | 40-4 W | 2.9 | 96.1 | 1.0 |
| F2: 20 mM Histidine, 8% Trehalose | T0 | 0.7 | 99.3 | NA |
| dihydrate, 0.02% PS80, pH 6.0 | 40-2 W | 2.3 | 97.3 | 0.4 |
| | 40-4 W | 2.9 | 96.1 | 0.9 |
| F3: 20 mM Histidine, 4% Sorbitol, | T0 | 0.6 | 99.4 | NA |
| 0.02% PS80, pH 6.0 | 40-2 W | 2.3 | 97.3 | 0.4 |
| | 40-4 W | 3.1 | 96.0 | 0.9 |
| F4: 20 mM Histidine, 4% Mannitol, | T0 | 1.4 | 98.6 | A |
| 0.02% PS80, pH 6.0 | 40-2 W | 2.7 | 96.9 | 0.4 |
| | 40-4 W | 3_2 | 95.8 | 1.0 |
| F5: 20 mM Histidine, 140 mM | T0 | 0.7 | 99.3 | NA |
| Arginine•HCl, 0.02% PS80, pH 6.0 | 40-2 W | 2.1 | 97.4 | 0.5 |
| | 40-4 W | 2.4 | 96.6 | 1.0 |
| F6: 20 mM Histidine, 8% Sucrose, | T0 | 0.6 | 99.4 | NA |
| pH 6.0 | 40-2 W | 1.3 | 98.2 | 0.5 |
| | 40-4 W | 1.9 | 97.2 | 1.0 |

SEC-I-IPLC results at 25° C. are shown in Table 15. These results indicated that there was no significant decrease in % Main peak after incubation at 25° C. for 4 weeks.

TABLE 15
SEC-HPLC Result of Excipient Screwing Study: 25° C.

| | | SEC-HPLC | | |
|---|---|---|---|---|
| | | | % | |
| | | % | Main | % |
| 25° C. | | HMW | peak | LMW |
| F1: 20 mM Histidine, 8% Sucrose, | T0 | 0.6 | 99.4 | NA |
| 0.02% PS80, pH 6.0 | 25-4 W | 1.2 | 98.6 | 0.3 |
| F2: 20 mM Histidine, 8% Trehalose | T0 | 0.7 | 99.3 | NA |
| dihydrate, 0.02% PS80, pH 6.0 | 25-4 W | 1.1 | 98.7 | 0.2 |
| F3: 20 mM Histidine, 4% Sorbitol, | T0 | 0.6 | 99.4 | NA |
| 0.02% PS80, pH 6.0 | 25-4 W | 1.0 | 98.8 | 0.2 |
| F4: 20 mM Histidine, 4% Mannitol, | T0 | 1.4 | 98.6 | NA |
| 0.02% PS80, pH 6.0 | 25-4 W | 1.6 | 98.2 | 0.2 |
| F5: 20 mM Histidine, 140 mM | T0 | 0.7 | 99.3 | NA |
| Arginine•HCl, 0.02% PS80, pH 6.0 | 25-4 W | 1.2 | 98.5 | 0.3 |
| F6: 20 mM Histidine, 8% Sucrose, | T0 | 0.6 | 99.4 | NA |
| pH 6.0 | 25-4 W | 0.9 | 99.0 | 0.2 |

SEC-HPLC results under freeze-thaw (−40° C. to RT) conditions are shown in Table 16 which show that there was no significant changes in these formulations after 10 cycles of freeze-thaw except F4 (mannitol, pH6.0), whose % Main peak decreased by 4.4% while % HMW increased by 4.4%.

TABLE 16
SEC-HPLC Result of Excipien1- Screening Study: Freeze-thaw

| | | SEC-HPLC | | |
|---|---|---|---|---|
| | | | % | |
| | | % | Main | % |
| Freeze thaw: −40° C. to RT | | HMW | peak | LMW |
| F1: 20 mM Histidine, 8% Sucrose, | T0 | 0.6 | 99.4 | NA |
| 0.02% PS80, pH 6:0 | 5 FT | 0.6 | 99.4 | NA |
| | 10 FT | 0.7 | 99.3 | NA |
| F2: 20 mM Histidine, 8% Trehalose | T0 | 0.7 | 99.3 | NA |
| dihydrate, 0.02% PS80, pH 6.0 | 5 FT | 0.7 | 99.3 | NA |
| | 10 FT | 0_7 | 99.3 | NA |
| F3: 20 mM Histidine, 4% Sorbitol, | T0 | 0.6 | 99.4 | NA |
| 0.02% PS80, pH 6.0 | 5 FT | 0.7 | 99.3 | NA |
| | 10 FT | 0.7 | 99.3 | NA |
| F4: 20 mM Histidine, 4% Mannitol, | T0 | 1.4 | 98.6 | NA |
| 0_0 2% PS80, pH 6.0 | 5 FT | 3.9 | 96.1 | NA |
| | 10 FT | 5.8 | 94.2 | NA |
| F5: 20 mM Histidine, 140 mM | T0 | 0.7 | 99.3 | NA |
| Arginine•HCl, 0.02% PS80, pH 6.0 | 5 FT | 0.7 | 99.3 | NA |
| | 10 FT | 0.7 | 99.3 | NA |
| F6: 20 mM Histidine, 8% Sucrose, | T0 | 0.6 | 99.4 | NA |
| pH 6.0 | 5 FT | 0.6 | 99.4 | NA |
| | 10 FT | 0.6 | 99.4 | NA |

### Caliper-SDS (nr)

Caliper-SDS is a CE-based high-throughput analysis method, which is used to detect fragmentation of monoclonal antibodies with high sensitivity. Caliper-SIDS (nr) results at 40° C. are shown in Table 17. % Purity reductions were observed in all 6 formulations. From F1 to F6, % purity was reduced by 5.8%, 3.7%, 4.4%, 4.9%, 4.8% and 4.6%, respectively. Among them, F2 (trehalose dihydrate, pH 6.0) showed the least % purity decline.

TABLE 17
Caliper-SDS (nr) Result of Excipient Screening Study: 40° C.

| | | Caliper-SDS(nr) |
|---|---|---|
| 40° C. | | % Purity |
| F1: 20 mM Histidine, 8% Sucrose, | T0 | 98.3 |
| 0.02% PS80, pH 6.0 | 40-2 W | 95.4 |
| | 40-4 W | 92.5 |
| F2: 20 mM Histidine, 8% Trehalose | T0 | 98.0 |
| dihydrate, 0.02% PS80, pH 6.0 | 40-2 W | 95.8 |
| | 40-4 W | 94.3 |
| F3: 20 mM Histidine, 4% Sorbitol, | T0 | 98.2 |
| 0.02% PS80, pH 6.0 | 40-2 W | 95.1 |
| | 40-4 W | 93.8 |
| F4: 20 mM Histidine, 4% Mannitol, | T0 | 98.2 |
| 0.02% PS80, pH 6.0 | 40-2 W | 95.6 |
| | 40-4 W | 93.3 |
| F5: 20 mM Histidine, 140 mM | T0 | 98.1 |
| Arginine•HCl, 0.02% PS80, pH 6.0 | 40-2 W | 95.4 |
| | 40-4 W | 93.3 |
| F6: 20 mM Histidine, 8% Sucrose, | T0 | 98.1 |
| pH 6.0 | 40-2 W | 95.9 |
| | 40-4 W | 93.5 |

Caliper-SDS (nr) results at 25° C. are shown in Table 18, The results indicated there was no significant change in these formulation after incubation at 25° C. for 4 weeks except F5 (Arginine·HCl, pH 6,0), whose % purity decreased by 1%.

TABLE 18
Caliper-SDS (nr) Result of Excipient Screening Study: 25° C.

| | | Caliper-SDS(nr) |
|---|---|---|
| 25° C. | | % Purity |
| F1: 20 mM Histidine, 8% Sucrose, | T0 | 98.3 |
| 0.02% PS80, pH 6,0 | 40-4 W | 97.7 |
| F2: 20 mM Histidine, 8% Trehalose | T0 | 98.0 |
| dihydrate, 0.02% PS80, pH 6.0 | 40-4 W | 97.6 |
| F3: 20 mM Histidine, 4% sorbitol, | T0 | 98.2 |
| 0.02% PS80, pH 6.0 | 40-4 W | 97.7 |
| F4: 20 mM Histidine, 4% Mannitol, | T0 | 98.2 |
| 0.02% PS80, pH 6.0 | 40-4 W | 97.9 |
| F5: 20 mM Histidine, 140 mM | T0 | 98.1 |
| Arginine•HCl, 0.02% PS80, pH 6.0 | 40-4 W | 97.1 |
| F6: 20 mM Histidine, 8% Sucrose, | T0 | 98.1 |
| pH 6.0 | 40-4 W | 97.9 |

Caliper-SDS (nr) results under freeze-thaw (−40° C. to RT) conditions are shown in Table 19. The results indicated that there was no significant change in % purity of all 6 formulations after 10 cycles of freeze-thaw.

TABLE 19
Caliper-SDS (nr) Result of Excipient Screening Study: Freeze-thaw

| | | Caliper-SDS(ru) |
|---|---|---|
| Freeze thaw: −40° C. to RT | | % Purity |
| F1: 20 mM Histidine, 8% Sucrose, | T0 | 98.3 |
| 0.02% PS80, pH 6.0 | 5 FT | 98.3 |
| | 10 FT | 98.1 |
| F2: 20 mM Histidine, 8% Trehalose | T0 | 98.0 |
| dihydrate, 0.02% PS80, pH 6.0 | 5 FT | 98.1 |
| | 10 FT | 97.9 |
| F3: 20 mM Histidine, 4% Sorbitol, | T0 | 98.2 |
| 0.02% PS80, pH 6.0 | 5 FT | 98.1 |
| | 10 FT | 98.2 |
| F4: 20 mM Histidine, 4% Mannitol, | T0 | 98.2 |
| 0.02% PS80, pH 6.0 | 5 FT | 98.1 |
| | 10 FT | 98.0 |
| F5: 20 mM Histidine, 140 mM | T0 | 98.1 |
| Arginine•HCl, 0.02% PS80, pH 6.0 | 5 FT | 98.1 |
| | 10 FT | 98.0 |
| F6: 20 mM Histidine, 8% Sucrose, | T0 | 98.1 |
| pH 6.0 | 5 FT | 98.1 |
| | 10 FT | 98.2 |

cIEF

The cIEF results at 40° C. are shown in Table 20. The increase in acidic peak and decrease in main peak were observed in all 6 formulations. From F1 to F6, % Main peak declined by 23.2%, 22.2%, 19.4%, 21.6%, 16.0% and 19.8%, respectively. And % Acidic peak increased by 24.8%, 21.6%, 18.6%, 21.0%, 18.0% and 18.0%, respectively. It's worth noting that there was no obvious change in % Basic peak.

TABLE 20
cIEF Result of Excipient Screening Study: 40° C.

| | | cIEF | | |
|---|---|---|---|---|
| | | % | % | % |
| | | Acidic | Main | Basic |
| 40° C. | | peak | peak | peak |
| F1: 20 mM Histidine, 8% Sucrose, | T0 | 16.2 | 60.7 | 23.1 |
| 0.02% PS80, pH 6.0 | 40-2 W | 29.1 | 48.1 | 22.8 |
| | 40-4 W | 41.0 | 37.5 | 21.5 |
| F2: 20 mM Histidine, 8% Trehalose | T0 | 17.3 | 61.6 | 21.2 |
| dihydrate, 0.02% , pH 6.0 | 40-2 W | 29.5 | 48.8 | 21.6 |
| | 40-4 W | 38.9 | 39.4 | 21.7 |
| F3: 20 mM Histidine, 4% Sorbitol, | T0 | 17.1 | 61.3 | 21.6 |
| 0.02% PS80, pH 6.0 | 40-2 W | 29.6 | 48.0 | 22.5 |
| | 40-4 W | 35.7 | 41.9 | 22.3 |
| F4: 20 mM Histidine, 4% Mannitol, | T0 | 18.3 | 60.0 | 21.7 |
| 0.02% PS80, pH 6.0 | 40-2 W | 29.7 | 48.4 | 21.9 |
| | 40-4 W | 39.3 | 38.4 | 22.3 |
| F5: 20 mM Histidine, 140 mM | T0 | 17.8 | 60.0 | 22.1 |
| Arginine•HCl, 0.02% PS80, pH 6.0 | 40-2 W | 27.9 | 50.7 | 21.4 |
| | 40-4 W | 35.8 | 44.0 | 20.2 |
| F6: 20 mM Histidine, 8% Sucrose, | T0 | 17.8 | 60.9 | 21.3 |
| pH 6.0 | 40-2 W | 26.0 | 50.6 | 23.5 |
| | 40-4 W | 35.8 | 41.1 | 23.1 |

After 4 weeks incubation at 25° C., the cIEF results are shown in Table 21. % Acidic peaks of 6 formulations were increased with the decrease of % Main peak. From F1 to F6, % Main peak declined by 5.8%, 5.4%, 7.5%, 5.4%, 5.2% and 4.3%, respectively; while % Acidic peaks increased by 6.8%, 4.2%, 7.3%, 4.5%, 5.8% and 2.9%, respectively.

TABLE 21
cIEF Result of Excipient Screening Study: 25° C.

| | | cIEF | | |
|---|---|---|---|---|
| | | % | % | % |
| | | Acidic | Main | Basic |
| 25° C. | | peak | peak | peak |
| F1: 20 mM Histidine, 8% Sucrose, | T0 | 16.2 | 60.7 | 23.1 |
| 0.02% PS80, pH 6.0 | 25-4 W | 23.0 | S4.9 | 22.2 |
| F2: 20 mM Histidine, 8% Trehalose | T0 | 17.3 | 61.6 | 21.2 |
| dihydrate, 0.02% PS80, pH 6.0 | 25-4 W | 21.5 | S6.2 | 22.3 |
| F3: 20 mM Histidine, 4% Sorbitol, | T0 | 17.1 | 61.3 | 21.6 |
| 0.02% PS80, pH 6.0 | 25-4 W | 24.4 | S3.8 | 21.8 |
| F4: 20 mM Histidine, 4% Mannitol, | T0 | 18.3 | 60.0 | 21.7 |
| 0.02% PS80, pH 6.0 | 25-4 W | 22.8 | S4.6 | 22.6 |
| F5: 20 mM Histidine, 140 mM | T0 | 17.8 | 60.0 | 22.1 |
| Arginine•HCl, 0.02% PS80, pH 6.0 | 25-4 W | 23.6 | 54.8 | 21.6 |
| F6: 20 mM Histidine, 8% Sucrose, | T0 | 17.8 | 60.9 | 21.3 |
| pH 6.0 | 25-4 W | 20.7 | 56.6 | 22.7 |

Table 22 shows the clEF results of the freeze-thaw experiments. The results indicated that the charge of 6 formulations did not change significantly after 10 cycles of freeze thaw.

TABLE 22
cIEF Result of Excipient Screening Study: Freeze-thaw

| | | cIEF | | |
|---|---|---|---|---|
| | | % | % | % |
| | | Acidic | Main | Basic |
| Freeze-thaw: −40° C. to RT | | peak | peak | peak |
| F1: 20 mM Histidine, 8% Sucrose, | T0 | 16.2 | 60.7 | 23.1 |
| 0.02% PS80, pH 6.0 | 5 FT | 18.2 | 59.9 | 21.9 |
| | 10 FT | 17.2 | 60.8 | 22.0 |
| F2: 20 mM Histidine, 8% Trehalose | T0 | 17.3 | 61.6 | 21.2 |
| dihydrate, 0.02% PS80, pH 6.0 | 5 FT | 17.2 | 61.3 | 21.5 |
| | 10 FT | 18.1 | 60.3 | 21.6 |
| F3: 20 mM Histidine, 4% Sorbitol, | T0 | 17.1 | 61.3 | 21.6 |
| 0.02% PS80, pH 6.0 | 5 FT | 18.1 | 60.6 | 21.3 |
| | 10 FT | 18.3 | 60.6 | 21.1 |
| F4: 20 mM Histidine, 4% Mannitol, | T0 | 18.3 | 60.0 | 21.7 |
| 0.02% PS80, pH 6.0 | 5 FT | 17.3 | 60.8 | 21.9 |
| | 10 FT | 17.5 | 61.3 | 21.2 |
| F5: 20 mM Histidine, 140 mM | T0 | 17.8 | 60.0 | 22.1 |
| Arginine•HCl, 0.02% PS80, pH 6.0 | 5 FT | 17.9 | 60.8 | 21.3 |
| | 10 FT | 17.8 | 60.8 | 21.4 |
| F6: 20 mM Histidine, 8% Sucrose, | T0 | 17.8 | 60.9 | 21.3 |
| pH 6.0 | 5 FT | 17.5 | 60.8 | 21.7 |
| | 10 FT | 17.4 | 61.0 | 21.6 |

### MFI

Sub-visible particles of each formulation were detected using the MFI 5200 Flow Microscope as shown in Table 23. MFI results showed, from Fl to F5, the number of particles (particle size ≥2 μm) at TO was in the same level. However, the number of particles (particle size ≥2 μm and particle size ≥10 μm) tested in F6 (without PS80) was much higher than that in other 5 formulations comparatively.

After 10 cycles of freeze-thaw, the number of particles (particle size ≥2 μm) in F3 (sorbitol, pH 6.0) increased from 1043/mL to 28428/mL, and the number of particles (particle size ≥10 μm) increased from 61/mL to 672/mL, respectively. In addition to this, no significant change was observed in other formulation samples.

TABLE 23
MFI Result of Excipient Screening Study: Freeze-thaw

| | | Sub-visible particle | | |
|---|---|---|---|---|
| | | (particle/mL) | | |
| | | ECD ≥ | ECD ≥ | ECD ≥ |
| Freeze-thaw: −40° C. to RT | | 2 μm | 10 μm | 25 μm |
| F1: 20 mM Histidine, 8% Sucrose, | T0 | 866 | 9 | 0 |
| 0.02% PS80, pH 6.0 | 10 FT | 328 | 12 | 0 |
| F2: 20 mM Histidine, 8% | T0 | 174 | 38 | 5 |
| Trehalose dihydrate, 0.02% PS80, | 10 FT | 238 | 15 | 5 |
| pH 6.0 | | | | |
| F3: 20 mM Histidine, 4% Sorbitol, | T0 | 1043 | 61 | 14 |
| 0.02% PS80, pH 6.0 | 10 FT | 28428 | 672 | 15 |
| F4: 20 mM Histidine, 4% | T0 | 976 | 40 | 4 |
| Mannitol, 0.02% PS80, pH 6.0 | 10 FT | 4090 | 166 | 22 |
| F5: 20 mM Histidine, 140 mM | T0 | 2096 | 68 | 4 |
| Arginine•HCl, 0.02% PS80, | 10 FT | 1679 | 25 | 0 |
| pH 6.0 | | | | |
| F6: 20 mM Histidine, 8% Sucrose, | T0 | 10448 | 507 | 15 |
| pH 6.0 | 10 FT | 5313 | 363 | 7 |

### Binding Antigen

Binding antigen method is an ELISA-based method used to detect the monoclonal antibody binding activity. The results are shown in Table 24. After 4 weeks of incubation at 25° C. and 40° C., there was no obvious decrease in the binding antigen activity of F2 (trehalose dihydrate, pH 6.0)

TABLE 24
Binding Antigen Result of Excipient Screening Study of F2

| | | Binding antigen |
|---|---|---|
| Formulation | Conditions | potency |
| F2: 20 mM Histidine, 8% Trehalose | T0 | 116% |
| dihydrate, 0.02% PS80, pH 6.0 | 25° C.-4 W | 115% |
| | 4 0° C.-4 W | 102% |

The stability of 6 formulations under 40° C., 25° C. and freeze-thaw (−40° C. to RT) conditions was investigated in this excipient screening study, After 4 weeks of incubation at 40° C., visible particles were observed in F6 (without PS80). Besides, the number of sub-visible particles tested in F6 at T0 was much higher than that in other formulations, which indicated that PS80 played a key role in inhibiting the aggregation of particles. SEC-HPLC results indicated that F3 (sorbitol, pH 6.0) showed the maximum decrease in % purity. In addition, caliper-SDS (non-reduced) results indicated that F1 (sucrose, pH 6.0) showed the most decline in % purity while F2 (trehalose, pH 6.0) showed the least. As shown in cIEF results, the most change was observed in F1 (sucrose, pH 6.0).

After 4 weeks of incubation at 25° C., no visible particles were observed in all 6 formulations. SEC-HPLC results showed that there was no obvious purity decline in 6 formulations. Caliper-SDS (non-reduced) results indicated that there was no significant changes in these formulation except FS (Arginine-HCl, pH 6.0). And clEF results showed that the charge of F3 (sorbitol, pH 6.0) changed the most.

In the experiment of freeze-thaw (−40° C. to RI), a large number of visible particles were observed in F4 (mannitol, pH 6.0) after 10 cycles of freeze-thaw, and SEC-HPLC results showed the Main peak of this formulation decreased by 4.4%. There was no significant changes in Caliper-SDS (non-reduced) and cIEF results in all these 6 formulations. MFI results manifested that the number of sub-visible particles of F3 (sorbitol, pH 6.0) were increased after 10 cycles of freeze-thaw.

Based on the above results, F1 (sucrose, pH 6.0), F3 (sorbitol, pH 6.0) and. F4 (mannitol, pH 6.0) showed poor performance. Caliper-SDS (non-reduced) results revealed that the % purity of F5 (Arginine-HCl, pH6.0) declined after 4 weeks incubation at 25° C. And DSC results showed that Tmₒₙₛₑₜ (° C.) value of F5 was at least 5° C. lower than that of other formulations. In conclusion, F2 (trehalose dihydrate, pH 6.0) performed better than other formulations. Therefore, F2 (trehalose dihydrate, pH 6.0) would be used in the subsequent surfactant screening study.

### Example 5. Surfactant Screening

This example screened for optimal concentrations of PS80 to stabilize IM005 protein.

The IM005 antibody (20.4 mg/mL) was exchanged into 20 mM histidine, pH6.0 buffer. The concentration of trehalose dihydrate was adjusted to 8% with its stock solution. Then, 10% stock solution of PS80 was added to make the final PS80 concentration to 0, 0.01%, 0.02% and 0,03%, respectively, as described in Table 25. The concentration of protein was adjusted to 50 mg/mL.

TABLE 25
Formulation for Surfactant Screening

| | | | | Surfactant | |
|---|---|---|---|---|---|
| No. | Protein | Buffer | Excipient | (PS 80) | pH |
| F1 | 50 mg/mL | 20 mM | 8% Trehalose | 0 | 6.0 |
| F2 | | Histidine, | dihydrate | 0.01% | |
| F3 | | pH 6.0 | | 0.02% | |
| F4 | | | | 0.03% | |

The prepared formulations were aseptically filtered with 022 μm PVDF membrane filter. For each formulation, 1 mL of sample was filled into 2 mL of glass vial, The vials were stoppered and crimped immediately after filling. All procedures were carried out in a bio-safety hood except for the crimping operation.

Vials were then placed in agitation condition (300 rmp and 25° C.). The samples were analyzed at the specified time points.

### Osmolality

The osmolality of F1-F4 at T0 was determined using an osmometer (Advanced 2020 Multi-Sample). Osmolality data are shown in Table 26.

TABLE 26
Osmolality Results at T0 of Surfactant Screening Study

| | | Osmolality |
|---|---|---|
| No. | Formulation | (mOsmol/kg) |
| F1 | 20 mM Histidine, 8% trehalose dihydrate, | 330 |
| | pH 6.0 | |
| F2 | 20 mM Histidine, 8% trehalose dihydrate, | 328 |
| | 0.01% PS80, pH 6.0 | |
| F3 | 20 mM Histidine, 8% trehalose dihydrate, | 327 |
| | 0.02% PS80, pH 6.0 | |
| F4 | 20 mM Histidine, 8% trehalose dihydrate, | 329 |
| | 0.03% PS80, pH 6.0 | |

### DSC

The thermodynamic stability of each formulations (F1-F4) at T0 was examined using DSC. As shown in Table 27, the onset transition temperatures of all formulations ranged from 56.30° C. to 57.39° C. The Tm1 value ranged from 64.39° C. to 65.23° C. and Tm2 value was in the range of 78.60° C. to 78.90° C. There was no significant difference in the thermodynamic transition profiles among the four formulations.

TABLE 27
DSC Results of Surfactant Screening Study

| | | Tm Onset | Tm1 | Tm2 |
|---|---|---|---|---|
| No. | Formulation | (0° C.) | (0° C.) | (0° C.) |
| F1 | 20 mM Histidine, 8% trehalose | 57.30 | 64.57 | 78.50 |
| | dihydrate, pH 6.0 | | | |
| F2 | 20 mM Histidine, 8% trehalose | 56.30 | 65.23 | 78.62 |
| | dihydrate, 0.01% PS80, pH 6.0 | | | |
| F3 | 20 mM Histidine, 8% trehalose | 57.39 | 65.22 | 78.60 |
| | dihydrate, 0.02% PS80, pH 6.0 | | | |
| F4 | 20 mM Histidine, 8% trehalose | 56.57 | 64.39 | 78.90 |
| | dihydrate, 0.03% PS80, pH 6.0 | | | |

### Appearance, pH, Protein Concentration

Visual inspection results are shown in Table 28. Plenty of visible particles were observed in F1 (without PS80) after agitation (300 rmp, 25° C.) for 1 day and 3 days. In contrast, no visible particle was observed in F2 (0.01% PS80), F3 (0.02% PS80) and F4 (0.03% PS80) under the same agitation conditions.

The pH values of F1-F4 were examined at all sampling points. As shown in Table 28, there was no significant change in pH value after agitation for 3 days at 25° C.

The protein concentrations of F1-F4 were measured using nanodrop2000 (UV280 method) at all sampling points. As shown in Table 28, no substantial change in protein concentration was detected after agitation for 3 days at 25° C.

TABLE 28
Appearance, pH and Concentration Results
of Surfactant Screening Study: Agitation

| Agitation: 25° C., | | | | UV280 |
|---|---|---|---|---|
| 300 rpm | | Appearance | pH | (mg/mL) |
| F1: 20 mM | T0 | Light yellow, slightly | 6.0 | 51.1 |
| Histidine, 8% | | opalescent, free of | | |
| trehalose | | visible particle | | |
| dihydrate, pH | 1 D | Many white visible particles | 6.1 | 49.8 |
| 6.0 | 3 D | Many white visible particles | 6.1 | 50.2 |
| F2: 20 mM | T0 | Light yellow, slightly | 6.0 | 50.8 |
| Histidine, 8% | | opalescent, free of | | |
| trehalose | | visible particle | | |
| dihydrate, 0.01% | 1 D | Light yellow, slightly | 6.1 | 51.6 |
| PS80, pH 6.0 | | opalescent, free of | | |
| | | visible particle | | |
| | 3 D | Light yellow, slightly | 6.1 | 50.3 |
| | | opalescent, free of | | |
| | | visible particle | | |
| F3: 20 mM | T0 | Light yellow, slightly | 6.0 | 50.1 |
| Histidine, 8% | | opalescent, free of | | |
| trehalose | | visible particle | | |
| dihydrate, 0.02% | 1 D | Light yellow, slightly | 6.1 | 50.3 |
| PS80, pH 6.0 | | opalescent, free of | | |
| | | visible particle | | |
| | 3 D | Light yellow, slightly | 6.1 | 50.4 |
| | | opalescent, free of | | |
| | | visible particle | | |
| F4: 20 mM | T0 | Light yellow, slightly | 6.0 | 49.9 |
| Histidine, 8% | | opalescent, free of | | |
| trehalose | | visible particle | | |
| dihydrate, 0.03% | 1 D | Light yellow, slightly | 6.1 | 50.6 |
| PS80, pH 6.0 | | opalescent, free of | | |
| | | visible particle | | |
| | 3 D | Light yellow, slightly | 6.1 | 50.2 |
| | | opalescent, free of | | |
| | | visible particle | | |

### SEC-HPLC

SEC-HPLC data are shown in Table 29. For F1-F4, no substantial change in % Main peak was observed after agitation for 3 days at 25° C.

TABLE 29
SEC-HPLC Results of Surfactant Screening Study: Agitation

| | | SEC-HPLC | | |
|---|---|---|---|---|
| | | | % | |
| | | % | Main | % |
| Agitation: 25° C., 300 rpm | | HMW | peak | LMW |
| F1: 20 mM Histidine, 8% trehalose | T0 | 0.6 | 99.4 | NA |
| dihydrate, pH 6.0 | 1 D | 0.6 | 99.4 | NA |
| | 3 D | 0.7 | 99.3 | NA |
| F2: 20 mM Histidine, 8% trehalose | T0 | 0.7 | 99.3 | NA |
| dihydrate, 0.01% PS80, pH 6.0 | 1 D | 0.7 | 99.3 | NA |
| | 3 D | 0.7 | 99.3 | NA |
| F3: 20 mM Histidine, 8% trehalose | T0 | 0.7 | 99.3 | NA |
| dihydrate, 0.02% PS80, pH 6.0 | 1 D | 0.7 | 99.3 | NA |
| | 3 D | 0.7 | 99.3 | NA |
| F4: 20 mM Histidine, 8% trehalose | T0 | 0.7 | 99.3 | NA |
| dihydrate, 0.03% PS80, pH 6.0 | 1 D | 0.7 | 99.3 | NA |
| | 3 D | 0.7 | 99.3 | NA |

### Caliper-SDS (nr)

Caliper-SDS is a microchip and CE based high-throughput analysis method, which is capable of detecting monoclonal antibodies fragments with high sensitivity. The results of Caliper-SDS (nr) are shown in Table 30. For all formulations (F1-F4), no substantial % purity reduction was observed after agitation for 3 days at 25° C.

TABLE 30
Caliper-SDS (nr) Results of Surfactant Screening Study: Agitation

| | | Caliper-SDS (nr) |
|---|---|---|
| Agitation: 25° C., 300 rpm | | % Purity |
| F1: 20 mM Histidine, 8% trehalose | T0 | 99.0 |
| dihydrate, pH 6.0 | 1 D | 99.0 |
| | 3 D | 99.0 |
| F2: 20 mM Histidine, 8% trehalose | T0 | 99.0 |
| dihydrate, 0.01% PS80, pH 6.0 | 1 D | 99.0 |
| | 3 D | 99.0 |
| F3: 20 mM Histidine, 8% trehalose | T0 | 99.0 |
| dihydrate, 0.02% PS80, pH 6.0 | 1 D | 98.9 |
| | 3 D | 98.9 |
| F4: 20 mM Histidine, 8% trehalose | T0 | 99.0 |
| dihydrate, 0.03% PS80, pH 6.0 | 1 D | 99.0 |
| | 3 D | 99.0 |

cIEF

cIEF data are shown in Table 31. For all formulations, no significant change in charge variants was observed after agitation for 3 days at 25° C.

TABLE 31
cIEF Results of Surfactant Screening Study: Agitation

| | | cIEF | | |
|---|---|---|---|---|
| | | % | % | % |
| | | Acidic | Main | Basic |
| Agitation: 25° C., 300 rpm | | peak | peak | peak |
| F1: 20 mM Histidine, 8% trehalose | T0 | 19.2 | 59.3 | 21.5 |
| dihydrate, pH 6.0 | 1 D | 20.5 | 57.2 | 22.3 |
| | 3 D | 20.2 | 57.6 | 22.2 |
| F2: 20 mM Histidine, 8% trehalose | T0 | 21.1 | 57.7 | 21.1 |
| dihydrate, 0.01% PS80, pH 6.0 | 1 D | 19.9 | 59.3 | 20.8 |
| | 3 D | 19.6 | 59.0 | 21.4 |
| F3: 20 mM Histidine, 8% trehalose | T0 | 20.8 | 58.2 | 21.0 |
| dihydrate, 0.02% PS80, pH 6.0 | 1 D | 20.4 | 57.9 | 21.7 |
| | 3 D | 21.3 | 57.0 | 21.7 |
| F4: 20 mM Histidine, 8% trehalose | T0 | 19.2 | 58.4 | 22.4 |
| dihydrate, 0.03% PS80, pH 6.0 | 1 D | 19.4 | 58.2 | 22.3 |
| | 3 D | 20.1 | 58.6 | 21.4 |

### MFI

Sub-visible particle detection was performed using MicroFlow image (MF15200). MEI data are shown in Table 29. At the sampling point of T0, the number of the particles with the particle size ≥25 μm was at the same level for F1-F4. However, the number of particles with the particle size ≥10 μm and ≥25 μm in F1 (without PS80) was much higher than other formulations (F2, F3 and F4).

After agitation for 3 days at 25° C., for F1 (without PS80), the number of particles with the particle size ≥2 μm increased from 715 particles/mL to 2585 particle/mL, ≥10 μm increased from 53 particle/mL to 579 particle/mL, and ≥25 μm increased from 4 particles/mL to 392 particles/mL, respectively. No significant difference was observed in F3 (0.02% PS80) and F4 (0.03% PS80) after agitation for 3 days. The number of particles with particles size ≥2 μm in F2 increased from 112 particles/mL to 1266 particles/mL, while the number of particles with particle size ≥10 μm and ≥25 μm did not change significantly.

TABLE 32
MFI Result of Surfactant Screening Study: Agitation.

| | | Sub-visible particle | | |
|---|---|---|---|---|
| | | (particle/mL) | | |
| | | ECD ≥ | ECD ≥ | ECD ≥ |
| Agitation: 25° C., 300 rpm | | 2 μm | 10 μm | 25 μm |
| F1: 20 mM Histidine, 8% trehalose | T0 | 715 | 53 | 4 |
| dihydrate, pH 6.0 | 1 D | 1297 | 38 | 15 |
| | 3 D | 2585 | 579 | 392 |
| F2: 20 mM Histidine, 8% trehalose | T0 | 112 | 5 | 4 |
| dihydrate, 0.0 I % PS80, pH 6.0 | 1 D | 369 | 14 | 4 |
| | 3 D | 1266 | 15 | 2 |
| F3: 20 mM Histidine, 8% trehalose | T0 | 217 | 15 | 5 |
| dihydrate, 0.02% PS80, pH 6.0 | 1 D | 174 | 9 | 2 |
| | 3 D | 243 | 2 | 7 |
| F4: 20 mM Histidine, 8% trehalose | T0 | 60 | 4 | 0 |
| dihydrate, 0.03% PS80, pH 6.0 | 1 D | 192 | 0 | 0 |
| | 3 D | 336 | 5 | 2 |

For all formulation candidates (F1-F4), no significant change was observed from the data of SEC-HPLC, Caliper-SDS (non-reduced) and cIEF after agitation at 300 rpm and 25° C. However, visible particles were observed in F1 (without PS80) through visual inspection after agitation for 1 day and 3 days, while no visible particle was observed in F2-F4 (with PS80 in different concentrations). As shown in MF:1 results, the number of sub-visible particles in F1 (without PS80) increased significantly, and the number of sub-visible particles with the particle size ≥2 μm in F2 (0.01% PS80) also increased, while no obvious change was detected in F3 (0.02% :PS80) and F4 (0.03% PS80).

The results therefore showed that PS80 played an important role in inhibiting particle generation under agitation conditions. However, PS80 is likely to degrade during long-term storage, which might reduce the stability of protein products. Therefore, lower PS80 concentration would be recommended when its capability of protein protection is guaranteed. Taken together, 0.02% was the finally selected concentration of PS80 for IM005.

Surfactant screening study indicated that PS80 played an important role in inhibiting particle generation and 0.02% PS80 was finally selected. Based on the results of both excipient and surfactant screening study, the formulation with 20 mM histidine, 8% trehalose dihydrate, 0.02% PS80 at pH 6.0 would be used in the final formulation confirmation study.

Excipient screening study was conducted by investigating five kinds of excipients under 40° C., 25° C. and freeze-thaw conditions, respectively. Formulation with trehalose dihydrate performed the best compared with other four excipients, thus, was selected for the subsequent surfactant screening study.

### Example 6, Formulation Confirmation

The study was conducted to confirm the lead formulation for IM005 as determined in Example 4. The lead formulation included 20 mM Histidine buffer, 8% (w/v) Trehalose Dihydrate, 0.02% (w/v) PS80, at pH6.0.

IM005 sample (51.7 mg/mL) was filtered with 0.22-μm filter, aseptically filled into 8 mL glass vials with 3.3 mL IM005 per vial, stoppered and sealed in a bio-safety hood.

### Appearance

Appearance of most samples are summarized in Table 33. All samples were slight yellow, slight opalescent liquid without visible particles. Only in 40° C.-2 W samples, a particle was observed, but no visible particle in 40° C.-4 W sample.

TABLE 33
Appearance evaluation of formulation confirmation

| | | | Sampling Points and Assay | | | |
|---|---|---|---|---|---|---|
| Condition | | T0 | 2 W | 4 W | SW | 12 W |
| 2~8° | C. | Light | / | Light | / | Light |
| | | yellow, | | yellow, | | yellow, |
| | | SO, | | SO, | | SO, |
| | | free of | | free of | | free of |
| | | particles | | particles | | particles |
| 25° | C. | | / | Light | Light | |
| | | | | yellow, | yellow, | |
| | | | | SO, | SO, | |
| | | | | free of | free of | |
| | | | | particles | particles | |
| 40° | c. | | Light | Light | | |
| | | | yellow, | yellow, | | |
| | | | SO, 1 | SO, | | |
| | | | particle | free of | | |
| | | | | particles | | |

SO = Slightly opalescent

### Sub-Visible Particle (MFI)

Counts of sub-visible particles in each sample were summarized in Table 34 and sub-visible particles were in control in all samples. Also, there is no obvious change of counts of sub-visible particle compared to T0 sample.

TABLE 34
Sub-visible particle results of formulation confirmation

| | | | Sampling Points and Assay | | | | | | | |
|---|---|---|---|---|---|---|---|---|---|---|
| | T0 | | 2 W | | 4 W | | 8 W | | 12 W | |
| Condition | ≥10 μm | ≥25 μm | ≥10 μm | ≥25 μm | ≥10 μm | ≥25 μm | ≥10 μm | ≥25 μm | ≥10 μm | ≥25 μm |
| 2~8° C. | 17 | 2 | / | / | 2 | 0 | / | / | 14 | 0 |
| 25° C. | | | / | / | / | / | 20 | 0 | / | / |
| 40° C. | | | / | / | 9 | 1 | / | / | / | / |

Unit = Counts/mL

### pH and Protein Concentration

pH and protein concentration results are summarized in Tables 35-36. pH and protein concentration of all samples were stable.

TABLE 35
pH results of formulation confirmation

Sampling Points and Assay

| | | | | | |
|---|---|---|---|---|---|
| Condition | T0 | 2 W | 4 W | 8 W | 12 W |

| | | | | | | |
|---|---|---|---|---|---|---|
| 2~8° | C. | 6.0 | / | 6.0 | / | 6.0 |
| 25° | C. | | / | 6.0 | 6.1 | / |
| 40° | c. | | 6.1 | 6.0 | / | / |

TABLE 36
Protein concentration results of formulation confirmation

Sampling Points and Assay

| | | | | | |
|---|---|---|---|---|---|
| Condition | T0 | 2 W | 4 W | 8 W | 12 W |

| | | | | | | |
|---|---|---|---|---|---|---|
| 2~8° | C. | 46.3 | / | 48.0 | 1 | 46.3 |
| 25° | C. | | / | 48.1 | 46.5 | / |
| 40° | c. | | 47.7 | 46.7 | / | / |

Unit = mg/mL

### SEC-HPLC

SEC results are summarized in Table 37. There was no obvious decrease of main peak of 2-8° C. samples. For 25° C. samples, main peak of 25° C.-8 W decreased by 1.0% and BMW increased by 0.8% compared to T0. For 40° C. samples, main peak of 40° C.-4 W decreased by 4.0% and HMW % increased by 3.5% compared to T0. This trend is consistent with results of previous formulation screening study.

TABLE 37
SEC results of formulation confirmation

| | | | | Sampling Points and Assay | | | | | |
|---|---|---|---|---|---|---|---|---|---|
| | T0 | | | 2 W | | | 4 W | | |
| Condition | Main | | | Main | | | Main | | |
| Area | Peak % | HMW % | LMW % | Peak % | HMW % | LMW % | Peak % | HMW % | LMW % |
| 2~8° C. | 98.5 | 1.5 | 0.0 | / | / | / | 98.4 | 1.6 | 0.0 |
| 25° C. | | | | / | / | / | 97.9 | 2.0 | 0.1 |
| 40° C. | | | | 96.6 | 3.0 | 0.3 | 94.5 | 5.0 | 0.6 |
| | | | | Sampling Points and Assay | | | | | |
| | T0 | | | 8 W | | | 12 W | | |
| Condition | Main | | | Main | | | Main | | |
| Area | Peak % | HMW % | LMW % | Peak % | HMW % | LMW % | Peak % | HMW % | LMW % |
| 2~8° C. | 98.5 | 1.5 | 0.0 | / | / | / | 98.2 | 1.7 | 0.1 |
| 25° C. | | | | 97.5 | 2.3 | 0.2 | / | / | / |
| 40° C. | | | | / | / | / | / | / | / |

### CE-SDS (r & nr)

CE-SDS reduced and non-reduced results were summarized in Tables 38-39. For CE-SDS reduced results, there was no obvious decrease of purity of 2-8° q samples. For 25° C. samples, purity of 25° C.-8W decreased by 0.6% compared to TO. For 40° C. samples, purity of 40° C.-4W decreased by 2.3% compared to TO. For CE-SDS non-reduced results, there was no obvious decrease of purity of 2-8° C. samples. For 25° C. samples, purity of 25° C.-8W decreased by 2.3% compared to T0. For 40° C. samples, purity of 40° C.-4 W decreased by 5.6% compared to T0. This trend is consist with results of previous formulation screening study.

TABLE 38
CE-SDS (reduced) results of formulation confirmation

| | | | | Sampling Points and Assay | | | | | |
|---|---|---|---|---|---|---|---|---|---|
| Condition | T0 | | | 2 W | | | 4 W | | |
| Area | HC % | LC % | Purity % | HC % | LC % | Purity % | HC % | LC % | Purity % |
| 2~8° C. | 33.5 | 64.8 | 98.3 | / | / | / | 33.6 | 64.9 | 98.5 |
| 25° C. | | | | / | / | / | 33.7 | 64.7 | 98.3 |
| 40° C. | | | | 33.5 | 63.6 | 97.1 | 33.4 | 62.6 | 96.0 |
| | | | | Sampling Points and Assay | | | | | |
| Condition | T0 | | | 8 W | | | 12 W | | |
| Area | HC % | LC % | Purity % | HC % | LC % | Purity % | HC % | LC % | Purity % |
| 2~8° C. | 33.5 | 64.8 | 98.3 | / | / | / | 32.8 | 65.5 | 98.3 |
| 25° C. | | | | 32.6 | 65.1 | 97.7 | / | / | / |
| 40° C. | | | | / | / | / | / | / | / |

TABLE 39
CE-SDS (non-reduced) results of formulation confirmation

| | | Sampling Points and Assay | | | |
|---|---|---|---|---|---|
| Condition | T0 | 2 W | 4 W | 8 W | 12 W |
| 2~8° C. | 98.2% | / | 97.8% | / | 97.9% |
| 25° C. | | / | 97.5% | 95.9% | / |
| 40° C. | | 94.9% | 92.6% | / | / |

cIEF

cIEF results are summarized in Table 40. There was no obvious decrease of main peak of 2-8° C.: samples. For 25° C.; samples, main peak of 25° C.-8 W decreased by 6.4% and acidic peak increased by 5.5% compared to T0. For 40° C. samples, main peak of 40° C.-4 W decreased by 22.5% and acidic peak increased by 16.4% compared to T0. This trend is consist with results of previous formulation screening study.

TABLE 40
cIEF results of formulation confirmation

| | | | | Sampling Points and Assay | | | | | |
|---|---|---|---|---|---|---|---|---|---|
| | T0 | | | 2 W | | | 4 W | | |
| Condition | Main | Acidic | Basic | Main | Acidic | Basic | Main | Acidic | Basic |
| Area | Peak % | Peak % | Peak % | Peak % | Peak % | Peak % | Peak % | Peak % | Peak % |
| 2~8° C. | 65.3 | 29.8 | 4.9 | I | I | I | 66.4 | 29.1 | 4.6 |
| 25° C. | | | | I | I | I | 62.6 | 31.2 | 6.1 |
| 40° C. | | | | 52.4 | 38.1 | 9.5 | / 42.8 | 46.2 | 11.0 |
| | | | | Sampling Points and Assay | | | | | |
| | T0 | | | 8 W | | | 12 W | | |
| Condition | Main | Acidic | Basic | Main | Acidic | Basic | Main | Acidic | Basic |
| Area | Peak % | Peak % | Peak % | Peak % | Peak % | Peak % | Peak % | Peak % | Peak % |
| 2~8° C. | 65.3 | 29.8 | 4.9 | / | / | / | 65.9 | 29.1 | 5.0 |
| 25° C. | | | | 58.9 | 35.3 | 5:9 | / | / | / |
| 40° C. | | | | / | / | / | / | / | / |

### Potency (Cell-Based Assay)

Potency results tested by cell-based assay are summarized in Table 41. There was no obvious change of potency (cell-based assay) of all tested samples during formulation confirmation. Increased of acidic peak of 25° C. and 40° C. samples didn't impact the potency.

TABLE 41
Potency results of formulation confirmation

Sampling Points and Assay

| | | | | | |
|---|---|---|---|---|---|
| Condition | T0 | 2 W | 4 W | 8 W | 12 W |

| | | | | | | |
|---|---|---|---|---|---|---|
| 2~8° | C. | 108% | / | 108% | / | 114% |
| 25° | c. | | / | / | 107% | / |
| 40° | c. | | / | 107% | / | / |

No obvious change was found in any of 2-8° C. samples in all test items. For accelerated condition 25° C. and stressful condition 40° C., the appearance, sub-visible particle, pH and protein concentration of samples can maintain consistent and stable. SEC-HPLC, CE-SDS (r & car) and clEF purity of these samples decreased to some extent, but the trend of decrease is consistent to that of the previous formulation development study.

The formulation of 20 mratlistidine buffer, 8% (w/v) Trehalose Dihydrate, 0.02% (w/v) PS80, at pH6.0 if therefore confirmed.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A composition comprising an anti-CD73 antibody, 5-50 mM histidine, 2%-20% (w/v) trehalose, and 0.015%-0.05% (w/v) polysorbate 80 (PS80), at pH 5.6-6.4, wherein the antibody comprises a heavy chain variable region (VH) comprising a CDR1, a CDR2 and a CDR3, and a light chain variable region (VL) comprising a CDR1, a CDR2 and a CDR3, and wherein the VH CDR1, VH CDR2, VH CDR3, VL CDR1, VL CDR2, and VL CDR3 comprise the amino acid sequences of SEQ ID NO:1-6, respectively.

2. The composition of claim 1, which comprises 10-30 mM histidine.

3. The composition of claim 1, which comprises 5%-15% (w/v) trehalose dihydrate.

4. The composition of claim 1, which comprises 0.015%-0.035% (w/v) PS80.

5. The composition of claim 1, which comprises 10-30 mM histidine, 5%-15% (w/v) trehalose, and 0.015%-0.035% (w/v) PS80, at pH 5.8-6.2.

6. The composition of claim 1, which comprises 15-25 mM histidine, 6%-10% (w/v) trehalose, 0.015%-0.025% (w/v) PS80, at pH 5.9-6.1.

7. The composition of claim 1, which comprises 5-150 mg/mL of the antibody.

8. The composition of claim 1, wherein the VH comprises the amino acid sequence of SEQ ID NO:7, and the VL comprises the amino acid sequence of SEQ ID NO:8.

9. The composition of claim 8, wherein the antibody comprises a heavy chain comprising the amino acid sequence of SEQ ID NO:9, and a light chain comprising the amino acid sequence of SEQ ID NO:10.

10. A lyophilized composition obtainable by freeze-drying the composition of claim 1.

11. A solution obtainable by dissolving the lyophilized composition of claim 10 in a solvent.

12. The solution of claim 11, wherein the solvent is water.

13. (canceled)

14. A method of treating cancer in a patient in need thereof, comprising administering to the patient the composition of claim 1.

15. The method of claim 14, wherein the cancer is selected from the group consisting of bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer.


SEQUENCE LISTING (10 sequences)

SEQ ID NO: 1 - protein, 6 aa, Artificial Sequence
  Synthetic
SGYYWN

SEQ ID NO: 2 - protein, 16 aa, Artificial Sequence
  Synthetic
YINYGGSNGYNPSLKS

SEQ ID NO: 3 - protein, 11 aa, Artificial Sequence
  Synthetic
DYDAYYEALDD

SEQ ID NO: 4 - protein, 10 aa, Artificial Sequence
  Synthetic
RASSRVNYMH

SEQ ID NO: 5 - protein, 7 aa, Artificial Sequence
  Synthetic
ATSNLAS

SEQ ID NO: 6 - protein, 9 aa, Artificial Sequence
  Synthetic
QQWSSNPPT

SEQ ID NO: 7 - protein, 120 aa, Artificial Sequence
  Synthetic
EVQLQESGPGLVKPSETLSLTCAVSGYSITSGYYWNWIRQPPGKKLEWMGYINYGGSNGYNPSLKSRITISRDTSKNQFSLKLSSVTAADTAVYYCARDYDAYYEALDDWGQGTTVTVSS

SEQ ID NO: 8 - protein, 106 aa, Artificial Sequence
  Synthetic
EIVLSQSPATLSLSPGERATLSCRASSRVNYMHWYQQKPGQSPRPWISATSNLASGVPARFSGSGSGTSYTLTISSLEPEDFAVYYCQQWSSNPPTFGGGTKVEIK

SEQ ID NO: 9 - protein, 450 aa, Artificial Sequence
  Synthetic
EVQLQESGPGLVKPSETLSLTCAVSGYSITSGYYWNWIRQPPGKKLEWMGYINYGGSNGYNPSLKSRITISRDTSKNQFSLKLSSVTAADTAVYYCARDYDAYYEALDDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, Artificial Sequence
  Synthetic
EIVLSQSPATLSLSPGERATLSCRASSRVNYMHWYQQKPGQSPRPWISATSNLASGVPARFSGSGSGTSYTLTISSLEPEDFAVYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC